(19) United States
(12) Patent Application Publication
(10) Pub. No.: US 2009/0054312 A1
(43) Pub. Date: Feb. 26, 2009

(54) SMIPs: Small molecule inhibitors of p27 depletion in cancers and other proliferative diseases

(76) Inventors: Dieter A. Wolf, La Jolla, CA (US); Gregory P. Roth, Oviedo, FL (US)

Correspondence Address: Biotechnology Law Group;c/o Portfolioip, P.O. Box 52050, Minneapolis, MN, 55402, US

(21) Appl. No.: 12/229,411

(22) Filed: Aug. 22, 2008

Related U.S. Application Data
(60) Provisional application No. 60/965,919, filed on Aug. 22, 2007.

(51) Int. Cl. A61K 38/13; C12Q 1/02; G01N 33/53; C07D 473/00; C07K 7/50; C07C 237/26; A61K 31/7076; A61K 31/65

(52) U.S. Cl. 514/11; 435/29; 435/7.2; 514/45; 514/152; 544/264; 552/203; 530/317

(57) ABSTRACT

Methods are provided for screening for SMIPs (small molecule inhibitors of p27 depletion). The SMIPs thus identified are useful for treating cancers and other proliferative diseases.

### CROSS-REFERENCE TO RELATED CASES

This application claims priority from U.S. provisional patent application Ser. No. 60/965,919, filed Aug. 22, 2007, which is incorporated herein by reference in its entirety.

### STATEMENT OF GOVERNMENT RIGHTS

The invention was supported, at least in part, by a grant from the Government of the United States of America (grant no. R01 CA116571 from the National Cancer Institute). The Government has certain rights to the invention.

### BACKGROUND

The cyclin-dependent kinase inhibitor p27 is required for an effective cell cycle arrest in vivo. This arrest is relieved by degradation of p27 via the ubiquitin-dependent proteolysis system. Depletion of the p27 tumor suppressor resulting from hyperproteolysis is a hallmark of advanced cancers (prostate, breast, colon, lung, melanoma, and others) that correlates with decreased survival. P27 proteolysis is mediated by SCF^{SKP2}-dependent polyubiquitination and subsequent proteasomal degradation. Many of these cancers overexpress SKP2, and SKP2 overexpression is sufficient to cause ectopic p27 degradation. Hyperactivation of the SKP2-mediated proteolysis pathway is therefore the principal mechanism of p27 downregulation in carcinomas.

### SUMMARY OF THE INVENTION

We have developed and deployed a screening assay to identify SMIPs, synthetic small molecules able to interfere with the depletion of the tumor suppressor p27 from human cancer cells (small molecule inhibitors of p27 depletion=SMIPs). Using this assay, we have screened 7368 chemical compounds from a variety of different libraries and identified 60 that show strong and reproducible SMIP activity. These compounds are useful for treating cancers and other proliferative diseases displaying p27 depletion.

According to one embodiment of the invention, methods are provided for identifying a small molecule inhibitor of p27 depletion comprising: (a) contacting a mammalian cell (for example, a human cell) with a test agent; and (b) determining whether the test agent inhibits ubiquitin-dependent proteolysis of p27 in the mammalian cell. In one embodiment, such a mammalian cell has substantially lower than normal levels of p27. In another embodiment, the mammalian cell overexpresses myc-SKP2, such as, for example, an LNCaP-S14 cell.

According to another embodiment, such methods comprise determining whether the test agent inhibits ubiquitin-dependent proteolysis of p27 in the mammalian cell by determining levels of p27 in the cell, including, but not limited to, by contacting the mammalian cell with an antibody that is specific for p27 and measuring binding of the antibody to p27 in the cell. In one such embodiment, for example, the antibody comprising a fluorophore and binding of the antibody to p27 in the cell is measured by measuring immunofluorescence of the mammalian cell.

According to another embodiment, such methods further comprise contacting a cancer cell with the test agent and determining whether the test agent inhibits ubiquitin-dependent proteolysis of p27 in the cancer cell (e.g., a human cancer cell), wherein the cancer cell is different than the mammalian cell. For example, in such embodiments, the cancer cell may be selected from the group consisting of a prostate cancer cell (e.g., an LNCaP-S14 cell), a cervical cancer cell (e.g., a HeLa cell), and a breast cancer cell (e.g., an MCF7 cell). In one such embodiment, such methods comprise determining whether the test agent inhibits ubiquitin-dependent proteolysis of p27 in the cancer cell by determining levels of p27 in the cancer cell. In another such embodiment, such methods comprise determining whether the test agent inhibits ubiquitin-dependent proteolysis of p27 in the cancer cell by inhibiting growth of the cancer cell or causing the death of the cancer cell.

In the foregoing methods, the mammalian cell may be provided, for example, in a well of a multi-well plate. Furthermore, any of the foregoing methods may be automated.

According to another embodiment of the invention, compounds are provided that inhibit ubiquitin-dependent proteolysis of p27 in a mammalian cell. According to one embodiment, such a compound is a compound of Formula I:

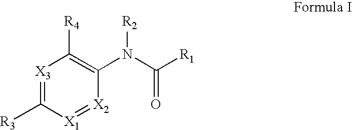

wherein:

R₁ is H, optionally substituted C1-C7 alkyl (linear, branched or cycloalkyl) optionally comprising one or more heteroatoms, wherein such heteroatoms are each O, S, or NRₓ, where R_{x is} C1-C7 alkyl, and also optionally comprising one or more unsaturated bonds (alkenyl or alkynyl) in the chain or ring;

R₂ is H, C1-C7 alkyl (linear, branched or cycloalkyl) optionally comprising one or more heteroatoms, wherein such heteroatoms are each O, S, or NRₓ, where R_{x is} C1-C7 alkyl, and also optionally comprising one or more unsaturated bonds (alkenyl or alkynyl) in the chain or ring; and wherein R₁ and R₂ optionally form a C4-C6 ring

R₃ is C1-C8 alkyl (linear, branched or cycloalkyl) optionally comprising one or more unsaturated bonds (alkenyl or alkynyl) in the chain or ring; C2-C8 aryl; or C2-C8 heteroaryl;

R4 is H, C1-C6 alkyl, or halo; and

X₁, X₂ and X₃ are each CH or N.

According to other embodiments, if one of R₁ or R₂ is H, then the other of R₁ or R₂ is optionally substituted alkyl (linear, branched or cycloalkyl) optionally comprising one or more heteroatoms, wherein such heteroatoms are each O, S, or NRₓ, where R_{x is} C1-C6 alkyl or C3-C7 cycloalkyl; and also optionally comprising one or more unsaturated bonds (alkenyl or alkynyl) in the chain or ring. For example, if R₁ is H, R₂ is optionally substituted alkyl (linear, branched or cycloalkyl) optionally comprising one or more heteroatoms (O, S, or NRₓ, where R_{x is} C1-C6 alkyl or C3-C7 cycloalkyl) and also optionally comprising one or more unsaturated bonds (alkenyl or alkynyl) in the chain or ring.

According to another embodiment, such a compound is a compound of Formula II:

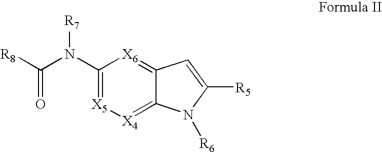

wherein:

R₅ is optionally substituted alkyl (linear, branched or cycloalkyl);

R₆ and R₇ are each H or Me;

R₈ is a five- or six-member optionally substituted aryl or optionally substituted heteroaryl; and

X₄, X₅ and X₆ are each CH or N.

According to another embodiment, SMIP compounds, or pharmaceutically acceptable prodrugs, salts or solvates thereof, according to the invention have structural formula III, which are analogs of SMIP 010:

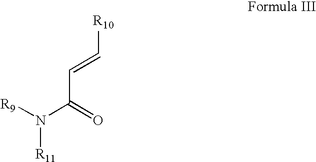

wherein:

R₉ is H or Me; and

R₁₀ and R₁₁ are each optionally substituted aryl or optionally substituted heteroaryl.

According to one embodiment, R₁₀ is optionally substituted phenyl. According to another embodiment, R₁₁ is optionally substituted phenyl or optionally substituted six-member monocyclic heteroaryl.

According to another embodiment, such a compound is a compound of Table 1.

According to another embodiment of the invention, a composition is provided comprising an effective amount of a compound of any of the aforementioned compounds and a pharmaceutically acceptable carrier.

According to another embodiment of the invention, methods are provided for inhibiting p27 depletion in a mammalian cell comprising contacting the mammalian cell with an effective amount of a composition comprising any of the aforementioned compounds.

According to another embodiment of the invention, methods are provided for treating a cancer in a patient in need of such treatment comprising administering to the patient an effective amount of a composition comprising any of the aforementioned compounds.

According to another embodiment of the invention, uses for any of the aforementioned compounds methods are provided to prepare a medicament to treat a cancer.

The foregoing and other aspects of the invention will become more apparent from the following detailed description, accompanying drawings, and the claims.

Unless otherwise defined, all technical and scientific terms used herein have the same meaning as commonly understood by one of ordinary skill in the art to which this invention pertains. Although methods and materials similar or equivalent to those described herein can be used in the practice or testing of the present invention, suitable methods and materials are described below.

### BRIEF DESCRIPTION OF THE FIGURES

FIG. 1 shows a summary of the results of the primary screening of 7368 compounds derived from a variety of different libraries (in duplicate) for compounds that upregulated p27 levels in LNCaP-S14 cells. The compounds were grouped into the categories weak, medium, and strong based on their z score: of the 176 hits in the primary screen, 60 compounds were strong (z>5), 58 were medium (z=3-5) and 58 were weak (z=2-3).

FIG. 2 shows the results of the secondary screening of 118 strong and medium compounds for compounds that upregulated p27 levels in LNCaP-S14, HeLa (cervical cancer), and MCF7 breast cancer cells. An image of a specific compound for each cell line is shown. Altogether, SMIP activity was confirmed for 67 compounds. 60 compounds were confirmed as active in LNCaP-S14 cells, 17 in HeLa cells, 46 in MCF7 cells. 15 compounds were specific for LNCaP-S14 cells, 1 for HeLa cells, and 5 for MCF7 cells. As shown, in the secondary screening, several compounds were active in more than one of the three cell types: 5 in both S14 and HeLa cells, 1 in both HeLa and Mcf7 cells, 30 in both S14 and Mcf7 cells, and 10 in all three cell types.

FIG. 3 shows SMIPs from various structural groups that were selected for further characterization: Amino indole series: SMIP001, SMIP003, SMIP011, SMIP013, SMIP014, SMIP015, SMIP016, SMIP017; Acetanylide: SMIP004; Diaryl urea: SMIP012; Tryptamine series: SMIP019, SMIP020, SMIP022; Cinnamic amide series: SMIP009, SMIP010; Thiadiazole: SMIP018; Aryl thiophene: SMIP002; Pyridazinone series: SMIP005, SMIP006; Aryl pyrazole: SMIP021.

FIG. 4 shows the accumulation of p27 upon treatment with SMIPs. S14 cells were treated with SMIPs (40 μM) for 16 hours and p27 levels were assayed by immunoblotting. The bar graph represents the average ±SD of two independent experiments normalized to tubulin.

FIG. 5 shows the effect of SMIPs on cell cycle distribution in LNCaP and S14 cells. Cells treated with 40 μM of the indicated SMIPs for 24 and 48 h were analyzed by flow cytometry.

FIG. 6 shows accumulation of p27 and p21 in S14 cells upon treatment with selected SMIPs. S14 cells were treated for 16 h with increasing concentrations of the respective SMIPs followed by immunoblotting for p27 and p21. A quantitation of duplicate immunoblotting experiments is shown.

FIG. 7 shows the effect of SMIPs on p27 stability in S14 cells. Cells were treated for 18 h with DMSO (0.1%), MG132 (20 μM) or SMIP001, 004 or 010 (40 μM). CHX (100 μg/ml) was added the next day and cell extracts were obtained at the time points indicated and analyzed by immunoblotting. Quantification of p27 levels normalized to time zero is shown.

FIG. 8 shows accumulation of p27 in prostate cancer cells upon treatment with SMIP004. S14 and parental LNCaP cells as well as cells of the prostate cancer cell line DU145 were treated with increasing concentrations of SMIP004 for 18 h. p27 levels were determined by immunoblotting and normalized to the tubulin control. The data shown is representative of two independent experiments.

FIG. 9 shows the effect of SMIP004 on p27 and SKP2 in S14 cells. S14 cells were treated with the indicated concentrations of SMIP004 for 18 h. Cytoplasmic and nuclear protein fractions were prepared and analyzed by immunoblotting with the indicated antibodies. Nucleolin and Tubulin signals served as nuclear and cytoplasmic markers. A quantification of the immunoblotting data is shown.

FIG. 10 shows quantitative real-time polymerase chain reaction (qRT-PCR) analysis of p27 and SKP2 mRNA in S14 cells. All data were normalized to b-actin and are expressed as arbitrary units (AU).

FIG. 11 shows analysis of six analogs of SMIP004, SMIP004_{—}1, SMIP004_{—}2, SMIP004_{—}3, SMIP004_{—}4, SMIP004_{—}5, and SMIP004_{—}6, which were tested with the immunofluorescence (IF) screening assay. LNCaP-S14 cells were treated with increasing concentrations (0-40 μM) of the analogs for 18 h. Cells were fixed and stained with p27 antibodies. The graph represents the average ±SD from 20 replicate wells per concentration.

FIG. 12 shows a general strategy for single-point analog and multi-point library synthesis.

FIG. 13 shows representative single-point analogs.

FIG. 14 shows examples of R1/R2 amide array components.

FIG. 15 shows an R3 and heteroatom substitution lipophilicity scan array.

FIG. 16 shows a conceptual diagram of a target directed SMIP004 affinity probe.

### DETAILED DESCRIPTION OF THE INVENTION

The present invention provides a method for screening for SMIPs (small molecule inhibitors of p27 depletion). The SMIPs thus identified are useful for treating cancers and other proliferative diseases, for example.

According to one embodiment of the invention, SMIP compounds, or pharmaceutically acceptable prodrugs, salts or solvates thereof, according to the invention have structural formula I, which are analogs of SMIP004:

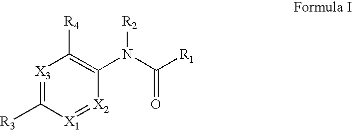

wherein:

R₁ is H, optionally substituted C1-C7 alkyl (linear, branched or cycloalkyl) optionally comprising one or more heteroatoms, wherein such heteroatoms are each O, S, or NRₓ, where R_{x is} C1-C7 alkyl, and also optionally comprising one or more unsaturated bonds (alkenyl or alkynyl) in the chain or ring;

R₂ is H, C1-C7 alkyl (linear, branched or cycloalkyl) optionally comprising one or more heteroatoms, wherein such heteroatoms are each O, S, or NRₓ, where R_{x is} C1-C7 alkyl, and also optionally comprising one or more unsaturated bonds (alkenyl or alkynyl) in the chain or ring; and wherein R₁ and R₂ optionally form a C4-C6 ring

R₃ is C1-C8 alkyl (linear, branched or cycloalkyl) optionally comprising one or more unsaturated bonds (alkenyl or alkynyl) in the chain or ring; C2-C8 aryl; or C2-C8 heteroaryl;

R4 is H, C1-C6 alkyl, or halo; and

X₁, X₂ and X₃ are each CH or N.

According to other embodiments, if R₁ or R₂ is H, then the other is optionally substituted alkyl (linear, branched or cycloalkyl) optionally comprising one or more heteroatoms, wherein such heteroatoms are each O, S, or NRₓ, where R_{x is} C1-C6 alkyl or C3-C7 cycloalkyl; and also optionally comprising one or more unsaturated bonds (alkenyl or alkynyl) in the chain or ring. For example, if R₁ is H, R₂ is optionally substituted alkyl (linear, branched or cycloalkyl) optionally comprising one or more heteroatoms (O, S, or NRₓ, where R_{x is} C1-C6 alkyl or C3-C7 cycloalkyl) and also optionally comprising one or more unsaturated bonds (alkenyl or alkynyl) in the chain or ring.

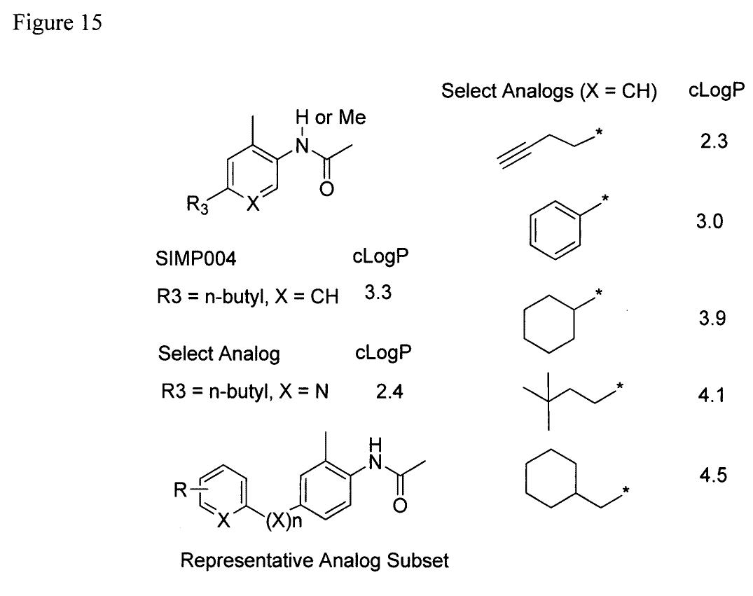

A number of analogs of SMIP004 have been found to have SMIP activity, as shown in FIG. 15.

According to another embodiment, SMIP compounds, or pharmaceutically acceptable prodrugs, salts or solvates thereof, according to the invention have structural formula II, which are analogs of SMIP001:

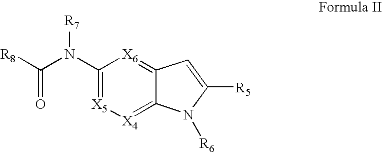

wherein:

R₅ is optionally substituted alkyl (linear, branched or cycloalkyl);

R₆ and R₇ are each H or Me;

R₈ is a five- or six-member optionally substituted aryl or optionally substituted heteroaryl; and

X₄, X₅ and X₆ are each CH or N.

According to another embodiment, SMIP compounds, or pharmaceutically acceptable prodrugs, salts or solvates thereof, according to the invention have structural formula III, which are analogs of SMIP010:

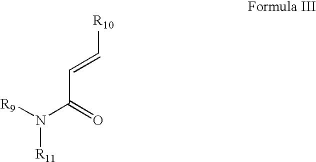

wherein:

R₉ is H or Me; and

R₁₀ and R₁₁ are each optionally substituted aryl or optionally substituted heteroaryl.

According to one embodiment, R₁₀ is optionally substituted phenyl. According to another embodiment, R₁₁ is optionally substituted phenyl or optionally substituted six-member monocyclic heteroaryl.

Additional SMIPs are included among the SMIP compounds of the present invention, including but not limited to the compounds listed in Table 1 and yet other compounds that are identified using the assays for SMIP activity provided herein.

### DEFINITIONS

As used herein, “SMIP” refers to any of the SMIPs discussed herein, including but not limited to the compounds of Formulae I, II and III, those listed in Table I below, and other SMIPs that are obtainable by screening according to the screening methods of the present invention, any analogs, and any pharmaceutically acceptable salt thereof.

As used herein, “agent” refers to any substance that has a desired biological activity. An “anti-cancer agent” has detectable biological activity in treating cancer, e.g., in killing a cancer cell, treating or preventing cancer, reducing or stopping growth of a cancer, or reducing a symptom of a cancer, in a host.

As used herein, “therapeutically effective amount” or simply “effective amount” refers to an amount of a composition that causes a detectable difference in an observable biological effect, for example, a statistically significant difference in such an effect. The detectable difference may result from a single substance in the composition, from a combination of substances in the composition, or from the combined effects of administration of more than one composition. For example, an “effective amount” of a composition comprising a SMIP of the present invention may refer to an amount of the composition that inhibits p27 depletion from a cell, such as, for example a cancer cell, or another desired effect, e.g., to treat or prevent a disease or disorder, or to treat the symptoms of a disease or disorder, in a host. A combination of a SMIP and another substance, e.g., an anti-cancer agent, or other active ingredient, in a given composition or treatment may be a synergistic combination. Synergy, as described for example by Chou and Talalay, Adv. Enzyme Regul. 22:27-55 (1984), occurs when the effect of the compounds when administered in combination is greater than the additive effect of the compounds when administered alone as a single agent. In general, a synergistic effect is most clearly demonstrated at suboptimal concentrations of the compounds. Synergy can be in terms of lower cytotoxicity, increased activity, or some other beneficial effect of the combination compared with the individual components.

As used herein, “treating” or “treat” includes (i) preventing a pathologic condition from occurring (e.g. prophylaxis); (ii) inhibiting the pathologic condition or arresting its development; (iii) relieving the pathologic condition; and/or diminishing symptoms associated with the pathologic condition.

As used herein, the term “patient” refers to organisms to be treated by the compositions and methods of the present invention. Such organisms include, but are not limited to, “mammals,” including, but not limited to, humans, monkeys, dogs, cats, horses, rats, mice, etc. In the context of the invention, the term “subject” generally refers to an individual who will receive or who has received treatment (e.g., administration of a compound of the invention, and optionally one or more anticancer agents) for cancer.

As used herein, “cells” refers to any animal cell, tissue, or whole organism, including but not limited to mammalian cells, e.g., bovine, rodent, e.g., mouse, rat, mink or hamster cells, equine, swine, caprine, ovine, feline, canine, simian or human cells.

As used herein, “pharmaceutically acceptable salts” refer to derivatives of a SMIP or other disclosed compounds wherein the parent compound is modified by making acid or base salts thereof. Examples of pharmaceutically acceptable salts include, but are not limited to, mineral or organic acid salts of basic residues such as amines; alkali or organic salts of acidic residues such as carboxylic acids; and the like. The pharmaceutically acceptable salts include the conventional non-toxic salts or the quaternary ammonium salts of the parent compound formed, for example, from non-toxic inorganic or organic acids. For example, such conventional non-toxic salts include those derived from inorganic acids such as hydrochloric, hydrobromic, sulfuric, sulfamic, phosphoric, nitric and the like; and the salts prepared from organic acids such as acetic, propionic, succinic, glycolic, stearic, lactic, malic, tartaric, citric, ascorbic, pamoic, maleic, hydroxymaleic, phenylacetic, glutamic, benzoic, salicylic, sulfanilic, 2-acetoxybenzoic, fumaric, toluenesulfonic, methanesulfonic, ethane disulfonic, oxalic, isethionic, and the like.

The pharmaceutically acceptable salts of a SMIP or other compounds useful in the present invention can be synthesized from the parent compound, which contains a basic or acidic moiety, by conventional chemical methods. Generally, such salts can be prepared by reacting the free acid or base forms of these compounds with a stoichiometric amount of the appropriate base or acid in water or in an organic solvent, or in a mixture of the two; generally, nonaqueous media like ether, ethyl acetate, ethanol, isopropanol, or acetonitrile are preferred. Lists of suitable salts are found in Remington's Pharmaceutical Sciences, 17th ed., Mack Publishing Company, Easton, Pa., p. 1418 (1985), the disclosure of which is hereby incorporated by reference.

The phrase “pharmaceutically acceptable” is employed herein to refer to those compounds, materials, compositions, and/or dosage forms which are, within the scope of sound medical judgment, suitable for use in contact with the tissues of human beings and animals without excessive toxicity, irritation, allergic response, or other problem or complication commensurate with a reasonable benefit/risk ratio.

One diastereomer of a compound disclosed herein may display superior activity compared with the other. When required, separation of the racemic material can be achieved by HPLC using a chiral column or by a resolution using a resolving agent such as camphonic chloride as in Thomas J. Tucker, et al., J. Med. Chem. 1994 37, 2437-2444. A chiral compound of Formula I may also be directly synthesized using a chiral catalyst or a chiral ligand, e.g. Mark A. Huffman, et al., J. Org. Chem. 1995, 60, 1590-1594.

“Stable compound” and “stable structure” are meant to indicate a compound that is sufficiently robust to survive isolation to a useful degree of purity from a reaction mixture, and formulation into an efficacious therapeutic agent. Only stable compounds are contemplated by the present invention.

“Substituted” is intended to indicate that one or more hydrogens on the atom indicated in the expression using “substituted” is replaced with a selection from the indicated group(s), provided that the indicated atom's normal valency is not exceeded, and that the substitution results in a stable compound. Suitable indicated groups include, e.g., alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxy. When a substituent is keto (i.e., ═O) or thioxo (i.e., ═S) group, then 2 hydrogens on the atom are replaced.

“Interrupted” is intended to indicate that in between two or more adjacent carbon atoms, and the hydrogen atoms to which they are attached (e.g., methyl (CH₃), methylene (CH₂) or methine (CH)), indicated in the expression using “interrupted” is inserted with a selection from the indicated group(s), provided that the each of the indicated atoms' normal valency is not exceeded, and that the interruption results in a stable compound. Such suitable indicated groups include, e.g., non-peroxide oxy (—O—), thio (—S—), carbonyl (—C(═O)—), carboxy (—C(═O)O—), imine (C═NH), sulfonyl (SO) or sulfoxide (SO₂).

Specific and preferred values listed below for radicals, substituents, and ranges, are for illustration only; they do not exclude other defined values or other values within defined ranges for the radicals and substituents

“Alkyl” refers to a C₁-C₁₈ hydrocarbon containing normal, secondary, tertiary or cyclic carbon atoms. Examples methyl (Me, —CH₃), ethyl (Et, —CH₂CH₃), 1-propyl (n-Pr, n-propyl, —CH₂CH₂CH₃), 2-propyl (i-Pr, i-propyl, —CH(CH₃)₂), 1-butyl (n-Bu, n-butyl, —CH₂CH₂CH₂CH₃), 2-methyl-1-propyl (i-Bu, i-butyl, —CH₂CH(CH₃)₂), 2-butyl (s-Bu, s-butyl, —CH(CH₃)CH₂CH₃), 2-methyl-2-propyl (t-Bu, t-butyl, —C(CH₃)₃), 1-pentyl (n-pentyl, —CH₂CH₂CH₂CH₂CH₃), 2-pentyl (—CH(CH₃)CH₂CH₂CH₃), 3-pentyl (—CH(CH₂CH₃)₂), 2-methyl-2-butyl (—C(CH₃)₂CH₂CH₃), 3-methyl-2-butyl (—CH(CH₃)CH(CH₃)₂), 3-methyl-1-butyl (—CH₂CH₂CH(CH₃)₂), 2-methyl-1-butyl (—CH₂CH(CH₃)CH₂CH₃), 1-hexyl (—CH₂CH₂CH₂CH₂CH₂CH₃), 2-hexyl (—CH(CH₃)CH₂CH₂CH₂CH₃), 3-hexyl (—CH(CH₂CH₃)(CH₂CH₂CH₃)), 2-methyl-2-pentyl (—C(CH₃)₂CH₂CH₂CH₃), 3-methyl-2-pentyl (—CH(CH₃)CH(CH₃)CH₂CH₃), 4-methyl-2-pentyl (—CH(CH₃)CH₂CH(CH₃)₂), 3-methyl-3-pentyl (—C(CH₃)(CH₂CH₃)₂), 2-methyl-3-pentyl (—CH(CH₂CH₃)CH(CH₃)₂), 2,3-dimethyl-2-butyl (—C(CH₃)₂CH(CH₃)₂), 3,3-dimethyl-2-butyl (—CH(CH₃)C(CH₃)₃, adamantyl, bridged bicyclic alkanes, and other linear, branched and cycloalkyl.

The alkyl can optionally be substituted with one or more alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. The alkyl can optionally be interrupted with one or more non-peroxide oxy (—O—), thio (—S—), carbonyl (—C(═O)—), carboxy (—C(═O)O—), sulfonyl (SO) or sulfoxide (SO₂). Additionally, the alkyl can optionally be at least partially unsaturated, thereby providing an alkenyl or alkynyl.

“Alkenyl” refers to a C₂-C₁₈ hydrocarbon containing normal, secondary, tertiary or cyclic carbon atoms with at least one site of unsaturation, i.e. a carbon-carbon, Sp² double bond. Examples include, but are not limited to: ethylene or vinyl (—CH═CH₂), allyl (—CH₂CH═CH₂), cyclopentenyl (—C₅H₇), and 5-hexenyl (—CH₂ CH₂CH₂CH₂CH═CH₂).

The alkenyl can optionally be substituted with one or more alkyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkenyl can optionally be interrupted with one or more non-peroxide oxy (—O—), thio (—S—), carbonyl (—C(═O)—), carboxy (—C(═O)O—), sulfonyl (SO) or sulfoxide (SO₂).

“Alkylidenyl” refers to a C₁-C₁₈ hydrocarbon containing normal, secondary, tertiary or cyclic carbon atoms. Examples are methylidenyl (═CH₂), ethylidenyl (═CHCH₃), 1-propylidenyl (═CHCH₂CH₃), 2-propylidenyl (═C(CH₃)₂), 1-butylidenyl (═CHCH₂CH₂CH₃), 2-methyl-1-propylidenyl (═CHCH(CH₃)₂), 2-butylidenyl (═C(CH₃)CH₂CH₃), 1-pentyl (═CHCH₂CH₂CH₂CH₃), 2-pentylidenyl (═C(CH₃)CH₂CH₂CH₃), 3-pentylidenyl (═C(CH₂CH₃)₂), 3-methyl-2-butylidenyl (═C(CH₃)CH(CH₃)₂), 3-methyl-1-butylidenyl (═CHCH₂CH(CH₃)₂), 2-methyl-1-butylidenyl (═CHCH(CH₃)CH₂CH₃), 1-hexylidenyl (═CHCH₂CH₂CH₂CH₂CH₃), 2-hexylidenyl (═C(CH₃)CH₂CH₂CH₂CH₃), 3-hexylidenyl (═C(CH₂CH₃)(CH₂CH₂CH₃)), 3-methyl-2-pentylidenyl (═C(CH₃)CH(CH₃)CH₂CH₃), 4-methyl-2-pentylidenyl (═C(CH₃)CH₂CH(CH₃)₂), 2-methyl-3-pentylidenyl (═C(CH₂CH₃)CH(CH₃)₂), and 3,3-dimethyl-2-butylidenyl (═C(CH₃)C(CH₃)₃.

The alkylidenyl can optionally be substituted with one or more alkyl, alkenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkylidenyl can optionally be interrupted with one or more non-peroxide oxy (—O—), thio (—S—), carbonyl (—C(═O)—), carboxy (—C(═O)O—), sulfonyl (SO) or sulfoxide (SO₂).

“Alkenylidenyl” refers to a C₂-C₁₈ hydrocarbon containing normal, secondary, tertiary or cyclic carbon atoms with at least one site of unsaturation, i.e. a carbon-carbon, sp² double bond. Examples include, but are not limited to: allylidenyl (═CHCH═CH₂), and 5-hexenylidenyl (═CHCH₂CH₂CH₂CH═CH₂).

The alkenylidenyl can optionally be substituted with one or more alkyl, alkenyl, alkylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkenylidenyl can optionally be interrupted with one or more non-peroxide oxy (—O—), thio (—S—), carbonyl (—C(═O)—), carboxy (—C(═O)O—), sulfonyl (SO) or sulfoxide (SO₂).

“Alkylene” refers to a saturated, branched or straight chain or cyclic hydrocarbon radical of 1-18 carbon atoms, and having two monovalent radical centers derived by the removal of two hydrogen atoms from the same or different carbon atoms of a parent alkane. Typical alkylene radicals include, but are not limited to: methylene (—CH₂—) 1,2-ethyl (—CH₂CH₂—), 1,3-propyl (—CH₂CH₂CH₂—), 1,4-butyl (—CH₂CH₂CH₂CH₂—), and the like.

The alkylene can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, the alkylene can optionally be interrupted with one or more non-peroxide oxy (—O—), thio (—S—), carbonyl (—C(═O)—), carboxy (—C(═O)O—), sulfonyl (SO) or sulfoxide (SO₂). Moreover, the alkylene can optionally be at least partially unsaturated, thereby providing an alkenylene.

“Alkenylene” refers to an unsaturated, branched or straight chain or cyclic hydrocarbon radical of 2-18 carbon atoms, and having two monovalent radical centers derived by the removal of two hydrogen atoms from the same or two different carbon atoms of a parent alkene. Typical alkenylene radicals include, but are not limited to: 1,2-ethylene (—CH═CH—).

The alkenylene can optionally be substituted with one or more alkyl, alkenyl, alkylidenyl, alkenylidenyl, alkoxy, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and/or COOR^{x}, wherein each R^{x} and R^{y} are independently H, alkyl, alkenyl, aryl, heteroaryl, heterocycle, cycloalkyl or hydroxyl. Additionally, The alkenylene can optionally be interrupted with one or more non-peroxide oxy (—O—), thio (—S—), carbonyl (—C(═O)—), carboxy (—C(═O)O—), sulfonyl (SO) or sulfoxide (SO₂).

The term “alkoxy” refers to the groups alkyl-O—, where alkyl is defined herein. Preferred alkoxy groups include, e.g., methoxy, ethoxy, n-propoxy, iso-propoxy, n-butoxy, tert-butoxy, sec-butoxy, n-pentoxy, n-hexoxy, 1,2-dimethylbutoxy, and the like.

The alkoxy can optionally be substituted with one or more alkyl, alkylidenyl, alkenylidenyl, halo, haloalkyl, hydroxy, hydroxyalkyl, aryl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “aryl” refers to an unsaturated aromatic carbocyclic group of from 6 to 20 carbon atoms having a single ring (e.g., phenyl) or multiple condensed (fused) rings, wherein at least one ring is aromatic (e.g., naphthyl, dihydrophenanthrenyl, fluorenyl, or anthryl). Preferred aryls include phenyl, naphthyl and the like.

The aryl can optionally be substituted with one or more alkyl, alkenyl, alkoxy, halo, haloalkyl, hydroxy, hydroxyalkyl, heteroaryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “cycloalkyl” refers to cyclic alkyl groups of from 3 to 20 carbon atoms having a single cyclic ring or multiple condensed rings. Such cycloalkyl groups include, by way of example, single ring structures such as cyclopropyl, cyclobutyl, cyclopentyl, cyclooctyl, and the like, or multiple ring structures such as adamantanyl, and the like.

The cycloalkyl can optionally be substituted with one or more alkyl, alkenyl, alkoxy, halo, haloalkyl, hydroxy, hydroxyalkyl, aryl, heteroaryl, heterocycle, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The cycloalkyl can optionally be at least partially unsaturated, thereby providing a cycloalkenyl.

The term “halo” refers to fluoro, chloro, bromo, and iodo. Similarly, the term “halogen” refers to fluorine, chlorine, bromine, and iodine.

“Haloalkyl” refers to alkyl as defined herein substituted by 1-4 halo groups as defined herein, which may be the same or different. Representative haloalkyl groups include, by way of example, trifluoromethyl, 3-fluorododecyl, 12,12,12-trifluorododecyl, 2-bromooctyl, 3-bromo-6-chloroheptyl, and the like.

The term “heteroaryl” is defined herein as a monocyclic, bicyclic, or tricyclic ring system containing one, two, or three aromatic rings and containing at least one nitrogen, oxygen, or sulfur atom in an aromatic ring, and which can be unsubstituted or substituted, for example, with one or more, and in particular one to three, substituents, like halo, alkyl, hydroxy, hydroxyalkyl, alkoxy, alkoxyalkyl, haloalkyl, nitro, amino, alkylamino, acylamino, alkylthio, alkylsulfinyl, and alkylsulfonyl. Examples of heteroaryl groups include, but are not limited to, 2H-pyrrolyl, 3H-indolyl, 4H-quinolizinyl, 4nH-carbazolyl, acridinyl, benzo[b]thienyl, benzothiazolyl, β-carbolinyl, carbazolyl, chromenyl, cinnaolinyl, dibenzo[b,d]furanyl, furazanyl, furyl, imidazolyl, imidizolyl, indazolyl, indolisinyl, indolyl, isobenzofuranyl, isoindolyl, isoquinolyl, isothiazolyl, isoxazolyl, naphthyridinyl, naptho[2,3-b], oxazolyl, perimidinyl, phenanthridinyl, phenanthrolinyl, phenarsazinyl, phenazinyl, phenothiazinyl, phenoxathiinyl, phenoxazinyl, phthalazinyl, pteridinyl, purinyl, pyranyl, pyrazinyl, pyrazolyl, pyridazinyl, pyridyl, pyrimidinyl, pyrimidinyl, pyrrolyl, quinazolinyl, quinolyl, quinoxalinyl, thiadiazolyl, thianthrenyl, thiazolyl, thienyl, triazolyl, and xanthenyl. In one embodiment the term “heteroaryl” denotes a monocyclic aromatic ring containing five or six ring atoms containing carbon and 1, 2, 3, or 4 heteroatoms independently selected from the group non-peroxide oxygen, sulfur, and N(Z) wherein Z is absent or is H, O, alkyl, phenyl or benzyl. In another embodiment heteroaryl denotes an ortho-fused bicyclic heterocycle of about eight to ten ring atoms derived therefrom, particularly a benz-derivative or one derived by fusing a propylene, or tetramethylene diradical thereto.

The heteroaryl can optionally be substituted with one or more alkyl, alkenyl, alkoxy, halo, haloalkyl, hydroxy, hydroxyalkyl, aryl, heterocycle, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

The term “heterocycle” refers to a saturated or partially unsaturated ring system, containing at least one heteroatom selected from the group oxygen, nitrogen, and sulfur, and optionally substituted with alkyl or C(═O)OR^{b}, wherein R^{b} is hydrogen or alkyl. Typically heterocycle is a monocyclic, bicyclic, or tricyclic group containing one or more heteroatoms selected from the group oxygen, nitrogen, and sulfur. A heterocycle group also can contain an oxo group (═O) attached to the ring. Non-limiting examples of heterocycle groups include 1,3-dihydrobenzofuran, 1,3-dioxolane, 1,4-dioxane, 1,4-dithiane, 2H-pyran, 2-pyrazoline, 4H-pyran, chromanyl, imidazolidinyl, imidazolinyl, indolinyl, isochromanyl, isoindolinyl, morpholine, piperazinyl, piperidine, piperidyl, pyrazolidine, pyrazolidinyl, pyrazolinyl, pyrrolidine, pyrroline, quinuclidine, and thiomorpholine.

The heterocycle can optionally be substituted with one or more alkyl, alkenyl, alkoxy, halo, haloalkyl, hydroxy, hydroxyalkyl, aryl, heteroaryl, cycloalkyl, alkanoyl, alkoxycarbonyl, amino, imino, alkylamino, acylamino, nitro, trifluoromethyl, trifluoromethoxy, carboxy, carboxyalkyl, keto, thioxo, alkylthio, alkylsulfinyl, alkylsulfonyl, cyano, NR^{x}R^{y} and COOR^{x}, wherein each R^{x} and R^{y} are independently H, alkyl, aryl, heteroaryl, heterocycle, cycloalkyl or hydroxyl.

Examples of nitrogen heterocycles and heteroaryls include, but are not limited to, pyrrole, imidazole, pyrazole, pyridine, pyrazine, pyrimidine, pyridazine, indolizine, isoindole, indole, indazole, purine, quinolizine, isoquinoline, quinoline, phthalazine, naphthylpyridine, quinoxaline, quinazoline, cinnoline, pteridine, carbazole, carboline, phenanthridine, acridine, phenanthroline, isothiazole, phenazine, isoxazole, phenoxazine, phenothiazine, imidazolidine, imidazoline, piperidine, piperazine, indoline, morpholino, piperidinyl, tetrahydrofuranyl, and the like as well as N-alkoxy-nitrogen containing heterocycles. In one specific embodiment of the invention, the nitrogen heterocycle can be 3-methyl-5,6-dihydro-4H-pyrazino[3,2,1-jk]carbazol-3-ium iodide.

Another class of heterocyclics is known as “crown compounds” which refers to a specific class of heterocyclic compounds having one or more repeating units of the formula [—(CH₂-)ₐA-] where a is equal to or greater than 2, and A at each separate occurrence can be O, N, S or P. Examples of crown compounds include, by way of example only, [—(CH₂)₃—NH—]₃, [—((CH₂)₂—O)₄—((CH₂)₂—NH)₂] and the like. Typically such crown compounds can have from 4 to 10 heteroatoms and 8 to 40 carbon atoms.

The term “alkanoyl” refers to C(═O)R, wherein R is an alkyl group as previously defined.

The term “acyloxy” refers to —O—C(═O)R, wherein R is an alkyl group as previously defined. Examples of acyloxy groups include, but are not limited to, acetoxy, propanoyloxy, butanoyloxy, and pentanoyloxy. Any alkyl group as defined above can be used to form an acyloxy group.

The term “alkoxycarbonyl” refers to C(═O)OR, wherein R is an alkyl group as previously defined.

The term “amino” refers to —NH₂, and the term “alkylamino” refers to —NR₂, wherein at least one R is alkyl and the second R is alkyl or hydrogen. The term “acylamino” refers to RC(═O)N, wherein R is alkyl or aryl.

The term “imino” refers to —C═NH.

The term “nitro” refers to —NO₂.

The term “trifluoromethyl” refers to —CF₃.

The term “trifluoromethoxy” refers to —OCF₃.

The term “cyano” refers to —CN.

The term “hydroxy” or “hydroxyl” refers to —OH.

The term “oxy” refers to —O—.

The term “thio” refers to —S—.

The term “thioxo” refers to (═S).

The term “keto” refers to (═O).

As to any of the above groups, which contain one or more substituents, it is understood, of course, that such groups do not contain any substitution or substitution patterns which are sterically impractical and/or synthetically non-feasible. In addition, the compounds of this invention include all stereochemical isomers arising from the substitution of these compounds.

Selected substituents within the compounds described herein are present to a recursive degree. In this context, “recursive substituent” means that a substituent may recite another instance of itself. Because of the recursive nature of such substituents, theoretically, a large number may be present in any given claim. One of ordinary skill in the art of medicinal chemistry understands that the total number of such substituents is reasonably limited by the desired properties of the compound intended. Such properties include, by of example and not limitation, physical properties such as molecular weight, solubility or log P, application properties such as activity against the intended target, and practical properties such as ease of synthesis.

Recursive substituents are an intended aspect of the invention. One of ordinary skill in the art of medicinal and organic chemistry understands the versatility of such substituents. To the degree that recursive substituents are present in a claim of the invention, the total number will be determined as set forth above.

The compounds described herein can be administered as the parent compound, a pro-drug of the parent compound, or an active metabolite of the parent compound.

“Pro-drugs” are intended to include any covalently bonded substances which release the active parent drug or other formulas or compounds of the present invention in vivo when such pro-drug is administered to a mammalian subject. Pro-drugs of a compound of the present invention are prepared by modifying functional groups present in the compound in such a way that the modifications are cleaved, either in routine manipulation in vivo, to the parent compound. Pro-drugs include compounds of the present invention wherein the carbonyl, carboxylic acid, hydroxy or amino group is bonded to any group that, when the pro-drug is administered to a mammalian subject, cleaves to form a free carbonyl, carboxylic acid, hydroxy or amino group. Examples of pro-drugs include, but are not limited to, acetate, formate and benzoate derivatives of alcohol and amine functional groups in the compounds of the present invention, and the like.

“Metabolite” refers to any substance resulting from biochemical processes by which living cells interact with the active parent drug or other formulas or compounds of the present invention in vivo, when such active parent drug or other formulas or compounds of the present are administered to a mammalian subject. Metabolites include products or intermediates from any metabolic pathway.

“Metabolic pathway” refers to a sequence of enzyme-mediated reactions that transform one compound to another and provide intermediates and energy for cellular functions. The metabolic pathway can be linear or cyclic.

### Pharmaceutical Compositions

The compounds of the invention can be formulated as pharmaceutical compositions and administered to a mammalian host, such as a human patient in a variety of forms adapted to the chosen route of administration, i.e., orally or parenterally, by intravenous, intramuscular, topical or subcutaneous routes.

The present compounds may be systemically administered, e.g., orally, in combination with a pharmaceutically acceptable vehicle such as an inert diluent or an assimilable edible carrier. They may be enclosed in hard or soft shell gelatin capsules, may be compressed into tablets, or may be incorporated directly with the food of the patient's diet. For oral administration, the active compound may be combined with one or more excipients and used in the form of ingestible tablets, buccal tablets, troches, capsules, elixirs, suspensions, syrups, wafers, and the like. Such compositions and preparations should contain at least 0.1% of active compound. The percentage of the compositions and preparations may, of course, be varied and may conveniently be between about 2 to about 60% of the weight of a given unit dosage form. The amount of active compound in such useful compositions is such that an effective dosage level will be obtained.

The tablets, troches, pills, capsules, and the like may also contain the following: binders such as gum tragacanth, acacia, corn starch or gelatin; excipients such as dicalcium phosphate; a disintegrating agent such as corn starch, potato starch, alginic acid and the like; a lubricant such as magnesium stearate; and a sweetening agent such as sucrose, fructose, lactose or aspartame or a flavoring agent such as peppermint, oil of wintergreen, or cherry flavoring may be added. When the unit dosage form is a capsule, it may contain, in addition to materials of the above type, a liquid carrier, such as a vegetable oil or a polyethylene glycol. Various other materials may be present as coatings or to otherwise modify the physical form of the solid unit dosage form. For instance, tablets, pills, or capsules may be coated with gelatin, wax, shellac or sugar and the like. A syrup or elixir may contain the active compound, sucrose or fructose as a sweetening agent, methyl and propylparabens as preservatives, a dye and flavoring such as cherry or orange flavor. Of course, any material used in preparing any unit dosage form should be pharmaceutically acceptable and substantially non-toxic in the amounts employed. In addition, the active compound may be incorporated into sustained-release preparations and devices.

The active compound may also be administered intravenously or intraperitoneally by infusion or injection. Solutions of the active compound or its salts can be prepared in water, optionally mixed with a nontoxic surfactant. Dispersions can also be prepared in glycerol, liquid polyethylene glycols, triacetin, and mixtures thereof and in oils. Under ordinary conditions of storage and use, these preparations contain a preservative to prevent the growth of microorganisms.

The pharmaceutical dosage forms suitable for injection or infusion can include sterile aqueous solutions or dispersions or sterile powders comprising the active ingredient which are adapted for the extemporaneous preparation of sterile injectable or infusible solutions or dispersions, optionally encapsulated in liposomes. In all cases, the ultimate dosage form should be sterile, fluid and stable under the conditions of manufacture and storage. The liquid carrier or vehicle can be a solvent or liquid dispersion medium comprising, for example, water, ethanol, a polyol (for example, glycerol, propylene glycol, liquid polyethylene glycols, and the like), vegetable oils, nontoxic glyceryl esters, and suitable mixtures thereof. The proper fluidity can be maintained, for example, by the formation of liposomes, by the maintenance of the required particle size in the case of dispersions or by the use of surfactants. The prevention of the action of microorganisms can be brought about by various antibacterial and antifungal agents, for example, parabens, chlorobutanol, phenol, sorbic acid, thimerosal, and the like. In many cases, it will be preferable to include isotonic agents, for example, sugars, buffers or sodium chloride. Prolonged absorption of the injectable compositions can be brought about by the use in the compositions of agents delaying absorption, for example, aluminum monostearate and gelatin.

Sterile injectable solutions are prepared by incorporating the active compound in the required amount in the appropriate solvent with various of the other ingredients enumerated above, as required, followed by filter sterilization. In the case of sterile powders for the preparation of sterile injectable solutions, the preferred methods of preparation are vacuum drying and the freeze drying techniques, which yield a powder of the active ingredient plus any additional desired ingredient present in the previously sterile-filtered solutions.

For topical administration, the present compounds may be applied in pure form, i.e., when they are liquids. However, it will generally be desirable to administer them to the skin as compositions or formulations, in combination with a dermatologically acceptable carrier, which may be a solid or a liquid.

Useful solid carriers include finely divided solids such as talc, clay, microcrystalline cellulose, silica, alumina and the like. Useful liquid carriers include water, alcohols or glycols or water-alcohol/glycol blends, in which the present compounds can be dissolved or dispersed at effective levels, optionally with the aid of non-toxic surfactants. Adjuvants such as fragrances and additional antimicrobial agents can be added to optimize the properties for a given use. The resultant liquid compositions can be applied from absorbent pads, used to impregnate bandages and other dressings, or sprayed onto the affected area using pump-type or aerosol sprayers.

Thickeners such as synthetic polymers, fatty acids, fatty acid salts and esters, fatty alcohols, modified celluloses or modified mineral materials can also be employed with liquid carriers to form spreadable pastes, gels, ointments, soaps, and the like, for application directly to the skin of the user.

Useful dosages of the compounds of the invention can be determined by comparing their in vitro activity and in vivo activity in animal models. Methods for the extrapolation of effective dosages in mice, and other animals, to humans are known to the art; for example, see U.S. Pat. No. 4,938,949.

Generally, the concentration of the compounds of the invention in a liquid composition, such as a lotion, will be from about 0.1-25 wt-%, preferably from about 0.5-10 wt-%. The concentration in a semi-solid or solid composition such as a gel or a powder will be about 0.1-5 wt-%, preferably about 0.5-2.5 wt-%.

The amount of the compound, or an active salt or derivative thereof, required for use alone or with other compounds will vary not only with the particular salt selected but also with the route of administration, the nature of the condition being treated and the age and condition of the patient and will be ultimately at the discretion of the attendant physician or clinician.

In general, however, a suitable dose may be in the range of from about 0.5 to about 100 mg/kg, e.g., from about 10 to about 75 mg/kg of body weight per day, such as 3 to about 50 mg per kilogram body weight of the recipient per day, preferably in the range of 6 to 90 mg/kg/day, most preferably in the range of 15 to 60 mg/kg/day.

The compound may be conveniently administered in unit dosage form; for example, containing 5 to 1000 mg, conveniently 10 to 750 mg, most conveniently, 50 to 500 mg of active ingredient per unit dosage form.

The active ingredient may be administered to achieve peak plasma concentrations of the active compound of from about 0.5 to about 75 μM, preferably, about 1 to 50 μM, most preferably, about 2 to about 30 μM. This may be achieved, for example, by the intravenous injection of a 0.05 to 5% solution of the active ingredient, optionally in saline, or orally administered as a bolus containing about 1-100 mg of the active ingredient. Desirable blood levels may be maintained by continuous infusion to provide about 0.01-5.0 mg/kg/hr or by intermittent infusions containing about 0.4-15 mg/kg of the active ingredient(s).

The desired dose may conveniently be presented in a single dose or as divided doses administered at appropriate intervals, for example, as two, three, four or more sub-doses per day. The sub-dose itself may be further divided, e.g., into a number of discrete loosely spaced administrations; such as multiple inhalations from an insufflator or by application of a plurality of drops into the eye.

Obviously, numerous modifications and variations of the present invention are possible in light of the above teachings. It is therefore to be understood that within the scope of the appended claims, the invention may be practiced otherwise than as specifically described herein.

The following invention will be further described by the following nonlimiting examples.

### EXAMPLE 1

The cyclin-dependent kinase inhibitor p27 is required for an effective cell cycle arrest in vivo. This arrest is relieved by degradation of p27 via the ubiquitin-dependent proteolysis system. Depletion of the p27 tumor suppressor resulting from hyperproteolysis is a hallmark of advanced cancers (prostate, breast, colon, lung, melanoma, and others) that correlates with decreased survival. P27 proteolysis is mediated by SCF^{SKP2}-dependent polyubiquitination and subsequent proteasomal degradation. Many of these cancers overexpress SKP2, and SKP2 overexpression is sufficient to cause ectopic p27 degradation. Hyperactivation of the SKP2-mediated proteolysis pathway is therefore the principal mechanism of p27 downregulation in carcinomas. This distinct point of vulnerability represents a thoroughly validated target for therapeutic intervention. Small molecules able to interfere with SKP2-dependent p27 degradation or to otherwise upregulate cellular p27 protein levels are predicted to inhibit the unrestrained growth or drive the death of cancer cells. Considering that virtually all normal differentiated cells express high levels of p27, such compounds bear a high potential for tumor specificity, thus minimizing drastic adverse effects. In addition, such compounds may be effective in treating other diseases, including but not limited to conditions involving angiogenesis (tumor, rheumatoid arthritis, psoriasis, vascular retinopathy, and endometriosis).

We have developed a cell-based screening system to identify SMIPs. We first developed a derivative of the LNCaP human prostate cancer cell line, in which maximal p27 proteolysis was driven by stable, ˜2-fold overexpression of Myc-SKP2 (=LNCaP-S14 cell line). To generate the LNCaP-S14 cell line, parental LNCaP cells were transfected with a plasmid driving the expression of c-Myc epitope-tagged SKP2 (pcDNA3.Myc-Skp2). Stable transfectants were selected in media containing geneticin, and individual drug resistant colonies were picked and propagated as permanent cell lines. LNCaP-S14 cells were derived from one of these colonies. These LNCaP-S14 cells have very low levels of p27, which can be restored upon the addition of proteasome inhibitors (MG132, epoxomycin). The cyclin-dependent kinase inhibitor roscovitine also upregulates p27 in S14 cells, a finding that is consistent with the known requirement of CDK2 activity for p27 degradation. The inhibition of p27 degradation by proteasome and kinase inhibitors was also apparent by conventional immunofluorescence staining. The immunostaining assay provided the basis for the cell-based screen described below.

Screening protocol. The immunofluorescence assay was adapted to 384-well plate format. All parameters, including the number of cells to be plated, fixation and blocking conditions, antibody concentrations, and incubation times with compounds were extensively optimized using positive (proteasome inhibitors, roscovitine) and negative controls (vehicle), resulting in the following reliable protocol: 4000 LNCaP-S14 cells per well were seeded in 25 μL RPMI, 10% FBS, using an automated liquid handler (Wellmate). After 24 h, 40-100 nL of positive control compound was transferred using a pin tool, followed by incubation for 16-18 h. Cells were fixed by adding 20 μL of 10% para-formaldehyde (final concentration 4%) directly to the medium and incubation for 20 minutes at room temperature. Cells were permeabilized by washing 3×5 min with PBS, 0.1% Triton X100 (30 μL/well), followed by blocking in 30 μL/well 5% nonfat dry milk in PBS, 0.1% Triton X100 for 1 hour at room temperature. Cells were incubated in 15 μl/well primary antibody (anti-p27, Transduction Laboratories K25030, 1:1000 in blocking buffer) for 1 h. Following one wash in 30 μL/well blocking buffer, 15 uL/well of secondary antibody (Alexa Fluor 594 goat anti-mouse, Invitrogen A11005, 1:200 in blocking buffer) was added for 1 h. Cells were washed 3×5 min in PBS, 0.1% Triton X100 (30 μL/well) and stained with Hoechst dye for two min at room temperature. Cells were washed twice in PBS, 5 min each. 30 μL/well of PBS were added, and plates were sealed and stored in the dark at 4° C. until scanning. All solutions were added using the Wellmate device, and aspirations were done with a 24 pin manual aspirator (“the wand”).

Plates were scanned in an ImageXpress automated microscope at dual wave length to detect Hoechst dye and Alexa Fluor 594. Two images per well were obtained at each wave length and analyzed with MetaXpress software. Pixel dimensions for detection of cell nuclei were set to 7×12, a setting that performed well in the detection of nuclear staining, while successfully excluding aggregates of cells growing on top of each other. Cells were scored positive for Hoechst dye if the integrated pixel intensity was 210-fold above local background, and positive for p27 when staining was 30-fold above background (signal obtained with secondary antibody). MetaXpress processing of the raw images provided quantitative measures of the total cell number and the number of p27 positive cells in a given field.

To evaluate the quality of our assay conditions, we determined the Z′-factor for the positive control reagent roscovitine. The Z′-factor measures the variation and separation bands of an assay and is defined as:

$${Z}^{′} = 1 - \frac{( 3 {SD}_{+} + 3 {SD}_{-} )}{\vert {Ave}_{+} - {Ave}_{-} \vert}$$

where “SD” refers to the standard deviation, “Ave” refers to the average staining intensity in all recorded wells, and “+” and “−” refer to positive and negative controls, respectively. We determined a Z′-factor of 0.48 for our assay, which is deemed appropriate for a successful screen.

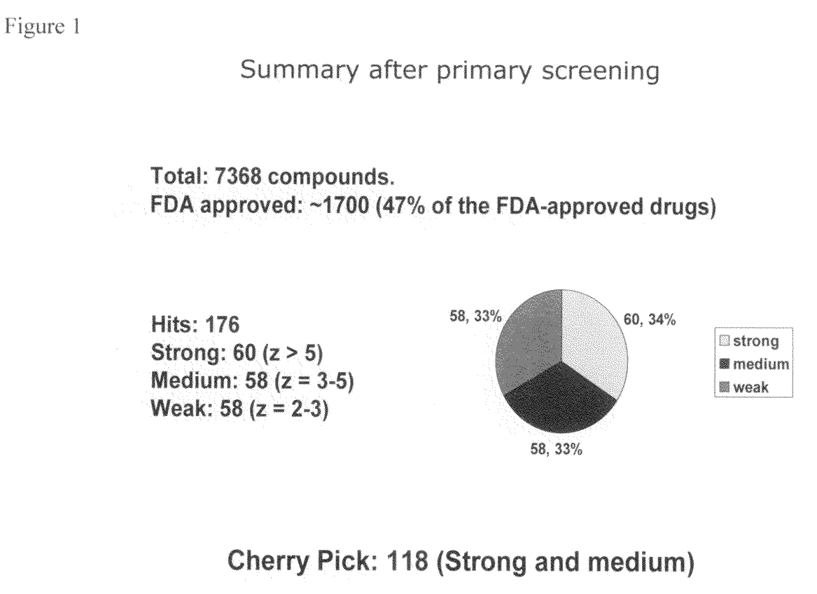

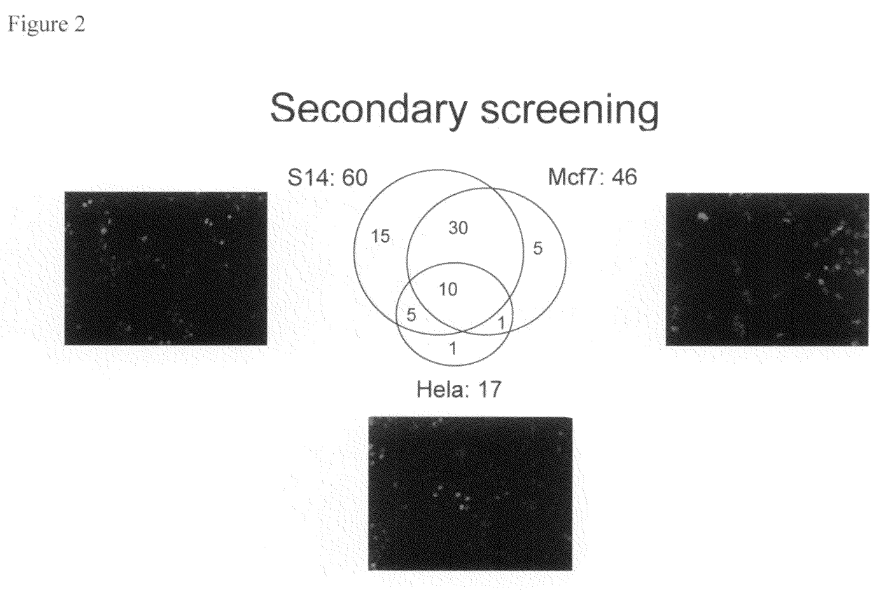

Screening results. Using the above protocol, we have so far screened 7,368 compounds derived from a variety of different libraries in duplicates. In the initial primary screen, we identified 176 compounds that upregulated p27 levels in LNCaP-S14 cells. The compounds were grouped into the categories weak, medium, strong based on their z score (FIG. 1). 118 strong and medium compounds were further examined by secondary screening in LNCaP-S14, HeLa (cervical cancer), and MCF7 (breast cancer) cells. Altogether, SMIP activity was confirmed for 67 compounds. 60 compounds were confirmed as active in LNCaP-S14 cells, 17 in HeLa cells, 46 in MCF7 cells. 15 compounds were specific for LNCaP-S14 cells, 1 for HeLa cells, and 5 for MCF7 cells (FIG. 2). The compounds in bold in Table 1 have been selected for further testing based on their favorable chemical properties (including but not limited to molecular weight, solubility, H-bond donor and acceptor count, etc.).

TABLE 1
Compounds showing SMIP activity in the secondary screen

| | % | | | | |
|---|---|---|---|---|---|
| Compound | positive | Z score | S14 | HeLa | Mcf7 |
| Puromycin hydrochloride | 63.07 | 42.4217 | x | | x |
| Salinomycin, sodium | 56.17 | 37.4159 | x | x | x |
| Clofilium tosylate | 53.49 | 35.4749 | x | x | x |
| 7-[(2-acetoxybenzoyl)amino]-1H-indole-2- | 51.60 | 34.1083 | x | | x |
| carboxylic acid, ethyl ester | | | | | |
| N-(4-butyl-2-methyl-phenyl)acetamide | 51.08 | 33.7296 | x | | |
| (SMIP004) | | | | | |
| Monensin, sodium salt (sodium 4-[2-[5- | 50.90 | 33.5974 | x | x | x |
| ethyl-5-[5-[6-hydroxy-6-(hydroxymethyl)- | | | | | |
| 3,5-dimethyl-oxan-2-yl]-3-methyl-oxolan- | | | | | |
| 2-yl]oxolan-2-yl]-9-hydroxy-2,8-dimethyl- | | | | | |
| 1,6-dioxaspiro[4.5]decan-7-yl)-3-methoxy- | | | | | |
| 2-methyl-pentanoate) | | | | | |
| Minocycline hydrochloride | 46.17 | 30.1696 | x | | |
| Lasalocid sodium | 45.21 | 29.4743 | x | | x |
| Mitomycin C | 42.52 | 27.5299 | x | | x |
| Niclosamide | 42.32 | 27.3834 | x | x | x |
| 3-[4-(methylsulfonyl)phenyl]-N-[3- | 41.56 | 26.8318 | x | | |
| trifluoromethyl]phenyl]-(2E)-propenamide | | | | | |
| Etoposide | 39.92 | 25.6406 | x | | x |
| N-[4-[[[4,6-bis(2,2,2- | 39.26 | 25.1654 | x | | x |
| trifluoroethoxy)pyrimidin-2- | | | | | |
| yl]methyl]thio]phenyl]-N′′-phenylurea | | | | | |
| 5-hexylsulfanyl-1,3,4-thiadiazol-2-amine | 38.52 | 24.6282 | x | | |
| Menadione (Vitamin K3) | 38.33 | 24.4943 | x | | x |
| N-(2-tert-butyl-(1H)-indol-5-yl)-4- | 37.42 | 23.8290 | x | x | x |
| chlorobenzenesulfonamide | | | | | |
| Camptothecin | 37.17 | 23.6514 | x | | x |
| Phenylmercuric acetate | 36.37 | 23.0742 | x | | x |
| Quinacrine hydrochloride | 36.17 | 22.9232 | x | | |
| Nigericin sodium | 35.05 | 22.1153 | x | x | |
| (4,5-dichloro-6-oxo-pyridazin-1- | 33.92 | 21.2964 | x | | |
| yl)methyl-4-(trifluoromethyl)benzoate | | | | | |
| N-(2-tert-butyl-(1H)-indol-5-yl)-N-(3- | 33.19 | 20.7674 | x | | x |
| chlorobenzyl)propanamide | | | | | |
| Spiperone | 32.90 | 20.5550 | x | | x |
| N-(4-bromo-2-methyl-phenyl)-4-(1H- | 30.52 | 18.8304 | x | | x |
| indol-3-yl)butanamide | | | | | |
| Dequalinium chloride | 30.24 | 18.6318 | x | | x |
| N-(2-tert-butyl-(1H)-indol-5-yl)-4- | 29.15 | 17.8415 | x | x | x |
| methoxybenzenesulfonamide | | | | | |
| Ciclopirox olamine | 28.54 | 17.3996 | x | | x |
| Oligomycin A (mix of A, B, C) | 26.65 | 16.0298 | x | x | x |
| Chlorhexidine hydrochloride | 25.83 | 15.4338 | x | | x |
| 1-(3-fluorophenyl)-2-[(5-pyridin-2-yl-4,5- | 25.37 | 15.1024 | x | | x |
| dihydro-1,3,4-thiadiazol-2- | | | | | |
| yl)sulfanyl]ethanone | | | | | |
| 4-[[(4-(4-chlorophenyl)thiophen-2- | 23.93 | 14.0559 | x | | |
| yl]carbonyl]-2,6-dimethylmorpholine | | | | | |
| (mixture of isomers) | | | | | |
| Parthenolide | 22.65 | 13.1286 | x | | x |
| Cyclosporin A | 20.78 | 11.7755 | x | | x |
| Sanguinarine nitrate | 20.35 | 11.4629 | x | | x |
| Sulconazole nitrate | 19.81 | 11.0719 | x | x | |
| (1R,2R,4S)-bicyclo[2.2.1]hept-2- | 19.39 | 10.7664 | x | | |
| ylmethyl[2-(5-methoxy-1H-indol-3- | | | | | |
| yl)ethyl]carbamate | | | | | |
| 2-(4-tert-butylbenzoyl)-6-methyl-1,2,3,4- | 19.38 | 10.7588 | x | | x |
| tetrahydro-11H-dipyrido-[1,2-a:4′,3′- | | | | | |
| d]pyrimidin-11-one | | | | | |
| Luteolin | 19.15 | 10.5904 | x | x | |
| Ethacrynic acid | 18.71 | 10.2724 | x | | x |
| Mefloquine | 17.66 | 9.5116 | x | x | x |
| Alexidine hydrochloride | 17.48 | 9.38 | x | | x |
| Securinine | 15.15 | 7.6953 | x | | x |
| Thiostrepton | 15.09 | 7.6485 | x | | x |
| N-(4-{[cyclopropyl(2,3-dihydro-1- | 14.84 | 7.4706 | x | x | |
| benzofuran-5- | | | | | |
| ylmethyl)amino]sulfonyl}phenyl)acetamide | | | | | |
| 4-(3,5-di-tert-butyl-1H-pyrazol-1-yl)-N- | 14.27 | 7.0595 | x | x | x |
| [(3S)-1-(2,4,6- | | | | | |
| trimethoxybenzyl)pyrrolidin-3- | | | | | |
| yl]benzamide | | | | | |
| 3-[4-(methylsulfonyl)phenyl]-N-(2- | 13.41 | 6.4339 | x | | x |
| pyridinyl)-(2E)-propenamide(SMIP010) | | | | | |
| N-(2-tert-butyl-(1H)-indol-5-yl)- | 13.27 | 6.3312 | x | | |
| thiophene-2-carboxamide (SMIP001) | | | | | |
| 3-chloro-2-[4-(morpholine-4-carbonyl)-1- | 12.42 | 5.7130 | x | x | x |
| piperidyl]naphthalene-1,4-dione | | | | | |
| Dichlorophene | 12.27 | 5.6065 | x | x | |
| Perphenazine | 12.07 | 5.4640 | x | | x |
| N-(4-chlorobenzyl)-7-[((4- | 11.30 | 4.9019 | x | | x |
| methoxyphenyl)sulfonyl)amino]-(1H)- | | | | | |
| indole-2-carboxamide | | | | | |
| Aclarubicin | 10.89 | 4.5990 | x | | x |
| 5-Azacytidine | 10.87 | 4.5963 | x | | x |
| Pyrithione zinc | 10.31 | 4.1877 | x | | x |
| 1-[2-(4-phenyl-1,3-thiazol-2-yl)ethyl]-3- | 10.24 | 4.1344 | x | | |
| (trifluoromethyl)pyridin-2(1H)-one | | | | | |
| Thonzonium | 9.65 | 3.7078 | x | | |
| N-(4-chlorobenzyl)-N-[((1H)-indol-6- | 9.23 | 3.4074 | x | | |
| yl)methyl]-4- | | | | | |
| methoxybenzenesulfonamide | | | | | |
| Methiothepin maleate | 9.08 | 3.2976 | x | | |
| Gossypol | 8.94 | 3.1932 | x | | |
| (4,5-dichloro-6-oxopyridazin-1(6H)- | 8.67 | 3.0023 | x | | |
| yl)methyl-3-(trifluoromethyl)benzoate | | | | | |
| Bepridil hydrochloride | 8.54 | 2.9020 | | x | x |
| N,N-diethyl-6-(4-fluorophenyl)-2-pyridin- | 4.69 | 0.1118 | | x | |
| 4-yl-pyrimidin-4-amine | | | | | |
| Fenbendazole | 4.46 | −0.0511 | | | x |
| Chlorprothixene hydrochloride | 7.49 | 2.1477 | | | x |
| Perhexilline maleate | 4.42 | −0.0837 | | | x |
| 5-methyl-4-(4-(trifluoromethyl)phenyl)- | 7.89 | 2.4371 | | | x |
| (1H)-imidazole | | | | | |
| Astemizole | 7.37 | 2.0598 | | | x |

### EXAMPLE 2

The cyclin-dependent kinase inhibitor p27 is required for an effective cell cycle arrest in vivo. This arrest is released by degradation of p27 via the ubiquitin-dependent proteolysis system. Depletion of the p27 tumor suppressor resulting from hyperproteolysis is a hallmark of many advanced cancers that correlates with decreased survival. Ubiquitin-mediated destruction of p27 is mediated by at least three different ubiquitin ligases (SCF^{SKP2}, KPC, PIRH2) all of which are overexpressed in prostate cancer. These enzymes are therefore attractive targets for therapeutic intervention. Small molecules able to interfere with p27 degradation are predicted to restore p27 expression in carcinomas, thus inhibiting their unrestrained growth. As discussed in Example 1, a phenotypic screen of 7,368 compounds was performed in a 384-well plate format to identify cell permeable compounds with the ability to upregulate p27 in prostate cancer cells overexpressing SKP2. 60 compounds (named SMIPs) effective with z scores of greater than 3 were identified and verified by secondary screening. Among those, SMIP004 upregulated p27 in various prostate cancer cell lines at low micromolar concentration, inhibited p27 degradation, preferentially killed prostate cancer cells over normal human fibroblasts, downregulated SKP2 protein and mRNA, and showed favorable pharmacological properties.

This Example presents the results of efforts to (1) optimize the potency of SMIP004 by medicinal chemistry and to provide a basic pharmacological characterization, (2) identify the cellular pathways and molecules targeted by SMIP004, and (3) test SMIP004 for its ability to upregulate p27 and inhibit prostate cancer cell growth in a mouse xenograft model.

### p27 Downregulation in Prevalent Human Carcinomas

A plethora of circumstantial evidence has implicated alterations in the level of the cyclin-dependent kinase (CDK) inhibitor p27 in prevalent carcinomas. For example, while more than 85% of terminally differentiated secretory cells in normal human prostate displayed strong nuclear staining for p27, all cases of high-grade prostatic intraepithelial neoplasia, invasive carcinoma, and pelvic lymph node metastases studied by DeMarzo et al. showed downregulation of p27 (Am. J. Pathol. 153:911-919, 1998). In addition, low p27 expression correlated with higher mean Gleason scores, positive surgical margins, seminal vesicle involvement, lymph node metastasis, aneuploidy, and a number of prognostic morphological features (Cheville et al., Mod. Pathol. 11:324-328, 1998). Finally, decreased p27 levels in prostate cancers were significantly associated with an increased probability of reoccurrence and decreased survival (Cote et al., J. Natl. Cancer Inst. 90:916-920, 1998). In contrast, ectopic expression of p27 can inhibit cell cycle progression in a human prostate cancer cell line (Kokontis et al., Mol. Endocrinol. 12:941-953, 1998), suppress astrocytoma growth in nude mice (Chen et al., J. Clin. Invest. 97:1983-1988, 1996), and induce the death of breast cancer cells (Craig et al., Oncogene 14:2283-2289, 1997). Other carcinomas displaying low p27 levels include breast cancer (Catzavelos et al., Nat. Med. 3:227-230, 1997; Fredersdorf et al., Proc. Natl. Acad. Sci. USA 94:6380-6385, 1997; Tan et al., Cancer Res. 57:1259-1263, 1997), gastric carcinoma (Masuda et al., Cancer Res. 62:3819-3825, 2002), small cell lung cancer (Yokoi et al., Am. J. Pathol. 161:207-216, 2002), oral squamous cell carcinoma (Gstaiger et al., Proc. Natl. Acad. Sci. USA 98:5043-5048, 2001; Kudo et al., Cancer Res. 61:7044-7047, 2001), and colon cancer (Hershko et al., Cancer 91:1745-1751, 2001), among several others (Slingerland and Pagano, J. Cell Physiol. 183:10-17, 2000). Based on these findings, p27 has been denoted as a tumor suppressor.

### Proteolytic Control of p27

In support of this notion are the findings that p27-deficient mice develop pituitary tumors with a high frequency (Fero et al., Nature 396:177-180, 1998; Cordon-Cardo et al., J. Natl. Cancer Inst. 90:1284-1291, 1998; Loda et al., Nat. Med. 3:231-234, 1997), and that the p27 protein is a target of viral oncoproteins (Pagano et al., Science 269:682-685, 1995; Hershko and Ciechanover, Ann. Rev. Biochem. 67:425-479, 1998). Remarkably, even heterozygous knockout mice are more sensitive to carcinogen-induced tumorigenesis (Fero et al., Nature 396:177-180, 1998). However, unlike traditional tumor suppressor genes, the p27 gene rarely shows homozygous inactivation in cancer cells (Tsvetkov et al., Curr. Biol. 9:661-664, 1999; Zheng et al., Nature 416:703-709, 2002; Hao et al., Molecular Cell 20:9-19, 2005), a finding that points towards alternative mechanisms of p27 inactivation. Similarly, many aggressive cancers display decreased p27 protein levels in the presence of high p27 mRNA (Catzavelos et al., Nat. Med. 3:227-230, 1997; Cordon-Cardo et al., J. Natl. Cancer Inst. 90:1284-1291, 1998), suggesting that p27 protein depletion result either from decreased mRNA translation or ectopic proteolysis. In fact, p27 protein depletion in colon cancer cells was shown to result from increased proteolysis via the ubiquitin/proteasome system (Loda et al., Nat. Med. 3:231-234, 1997).

### Ubiquitin-Dependent Proteolysis of p27

p27 accumulates in cells when the ubiquitin/proteasome-dependent proteolysis system is inhibited (Pagano et al., Science 269:682-685, 1995). This system employs a cascade of enzymatic reactions that covalently attach a chain of multiple ubiquitins to a substrate protein (Hershko and Ciechanover, Ann. Rev. Biochem. 67:425-479, 1998). Modification by ubiquitin serves as a molecular “tag” that is recognized by the proteasome, ultimately resulting in protein degradation. The ubiquitin transfer reaction involves three enzymes: E1, which mediates the ATP-dependent activation of ubiquitin, an E2, or ubiquitin conjugating enzyme, which, together with an E3, transfers ubiquitin to the target protein. The E3 enzyme, which is also called ubiquitin ligase, is particularly significant because it is directly involved in substrate binding.

### p27 Proteolysis is Mediated by at Least Three Different Ubiquitin Ligases: SCF^{SKP2}, KPC, and PIRH2

It has become increasingly evident that many proteins underlie complex stability control by different E3s. A point in case is p53, for which at least six E3s are known (E6-AP, HDM2, COP1, PIRH2, WWP1, and ARF-BP1). Likewise for p27, three E3s have been identified that control p27 stability in different cell cycle phases and at different cellular locales (summarized in Table 2).

TABLE 2
E3 ubiquitin ligases targeting p27

| | Cell cycle | | | Overexpressed |
|---|---|---|---|---|
| E3 | phase | Localization | Phosphorylation | in PCa |
| KPC | G0, G1 | Nuclear, | ? | Yes (KPC2) |
| | | cytoplasmic | | |
| PIRH2 | G1, S | Cytoplasmic | S10 | Yes |
| SCF^{SKP2} | S, G2 | Nuclear | T187 | Yes (SKP2) |

SCF^{SKP2}. Biochemical studies identified SKP2 as a subunit of a ubiquitin ligase complex, SCF^{SKP2}, that mediates p27 degradation at the beginning of the S phase (Carrano et al., Nature Cell Biol. 1:193-199, 1999; Tsvetkov et al., Curr. Biol. 9:661-664, 1999) and performs p27 ubiquitylation in vitro (Carrano et al., Nature Cell Biol. 1:193-199, 1999; Tsvetkov et al., Curr. Biol. 9:661-664, 1999). This complex was crystallized (Zheng et al., Nature 416:703-709, 2002; Hao et al., Molecular Cell 20:9-19, 2005) and consists of five proteins: SKP1, CUL1, RBX1 (═HRT1/ROC1), CKS1, and SKP2. SKP2 contains a so-called F-box which mediates binding to SKP 1, and C-terminal leucine-rich repeats that recognize p27 in complex with CDK2 and cyclin E. CUL1, in turn binds to SKP1, and together with RBX1, mediates the interaction with the ubiquitin-conjugating enzyme CDC34/UBC3. Notably, p27 requires phosphorylation at threonine 187 by cyclin-dependent kinase 2 (CDK2) in order to interact with SKP2 (Lu et al., BMC Cell Biol. 3:22, 2002; Shim et al., Cancer Res. 63:1583-1588, 2003; Cardozo and Pagano, BMC Biochem. 8 (Suppl. 1):S9, 2007; Nalepa et al., Nat. Rev. Drug Discov. 5:596-613, 2006; Richardson et al., Annu. Rev. Med. 57:33-47, 2006). CKS1 is a small protein that associates with CDK2 and greatly stimulates p27 binding to SCF^{SKP2} and its subsequent ubiquitylation (Ganoth et al., Nat. Cell Biol. 3:321-324, 2001; Spruck et al., Mol. Cell. 7:639-650, 2001). In summary, SCF^{SKP2}-mediated p27 degradation is the result of complex multiprotein interactions that appear to be disturbed in cancer cells lacking p27 expression.

KPC. Whereas SCF^{SKP2} is thought to mediate the degradation of nuclear p27 throughout the S phase and G2, another ubiquitin ligase, the heterodimeric KPC complex, targets p27 for cytoplasmic degradation upon cell cycle entry from G0 and during the G1 phase (Dreicer et al., Clin. Cancer Res. 13:1208-1215, 2007). Although somewhat controversial (Kuruvilla et al., Nature 416:653-657, 2002), it is thought that KPC targets p27 upon nuclear export that is mediated by phosphorylation on S10 (Stockwell et al., Chem. Biol. 6:71-83, 1999; Yang et al., Clin. Cancer Res. 8:3419-3426, 2002; Sheaff et al., Genes Dev. 11:1464-1478) and perhaps T157 (Vlach et al., EMBO J. 16:5334-5344, 1997). These modifications are mediated by a variety of kinases, including MIRK, hKIS, and AKT.

KPC1 is a RING domain E3 (also known as RNF123), whereas KPC2 contains UBL and UBA domains found in many UPS proteins. While overexpression of KPC1 and KPC2 accelerates p27 degradation upon cell cycle entry of NIH3T3 cells, knockdown of KPC does not slow the progression of MEFs into the S phase (Dreicer et al., Clin. Cancer Res. 13:1208-1215, 2007). However, simultaneous depletion of both KPC and SKP2 leads to cell cycle delay (Kamura et al., Nat. Cell Biol. 6:1229-1235, 2004). Thus, KPC and SKP2 appear to cooperate in p27 degradation and cell cycle progression.

PIRH2. The RING domain ligase PIHR2 is the most recently identified p27 E3 (Hattori et al., Cancer Res. 67:10789-10795, 2007). Like SCF^{SKP2} and KPC, PIRH2 interacts with p27 and ubiquitinates the protein in vitro and in vivo. A comparative knockdown of SKP2, KPC, and PIRH2 in T98G glioblastoma cells revealed approximately equally strong effects on p27. However, unlike the other E3s, PIRH2 targets both nuclear and cytoplasmic p27 (Hattori et al., Cancer Res. 67:10789-10795, 2007). The same study also demonstrated that only PIRH2 knockdown, but not depletion of SKP2 or KPC, slows cell cycle progression in T98G cells. The generality of this finding is still unclear as no other cell lines were examined. In addition, the comparison was hampered by the differing knockdown efficiencies for the various E3s, which varied between 50 and 75% (Hattori et al., Cancer Res. 67:10789-10795, 2007). The authors concluded that all three ligases coordinately control p27 stability.

### p27 Proteolysis Pathways as Potential Cancer Drug Targets

Among the three p27 proteolysis pathways, SKP2 is most extensively studied with respect to its role in tumorigenesis. SKP2 overexpression is frequent in human carcinomas devoid of p27 expression (Masuda et al., Cancer Res. 62:3819-3825, 2002; Yokoi et al., Am. J. Pathol. 161:207-216, 2002; Gstaiger et al., Proc. Natl. Acad. Sci. USA 98:5043-5048, 2001; Kudo et al., Cancer Res. 61:7044-7047, 2001; Hershko et al., Cancer 91:1745-1751, 2001; Zhang et al., J. Biomol. Screen. 4:67-73, 1999; Zhang and Kaelin, Meth. Enzymol. 399:530-549, 2005). In addition, our own data have shown that SKP2 overexpression in LNCaP prostate cancer cells is sufficient to mediate p27 ubiquitylation and degradation (Lu et al., BMC Cell Biol. 3:22, 2002). Furthermore, transgenic expression of SKP2 in mouse prostate causes dysplasia and low-grade prostate carcinomas that coincide with p27 downregulation (Shim et al., Cancer Res. 63:1583-1588, 2003). Conversely, RNAi-mediated knockdown of SKP2 expression inhibits tumor growth in a mouse transplant model (Sumimoto et al., Gene Ther., 2004). SKP2⁻/⁻ knockout mice are small and display general organ hypoplasia. This phenotype was fully rescued by simultaneous deletion of p27 in SKP2⁻/⁻ p27⁻/⁻ double knockout mice (Nakayama et al, Dev. Cell 6:661-672, 2004). Thus, decreasing the E3 activity of SKP2 mostly affects cell growth by allowing the accumulation of p27. These findings validate SKP2 (and its associated E3 complex) as a formidable cancer drug target (Cardozo and Pagano, BMC Biochem 8 [Suppl. 1]:S9, 2007). Considering that virtually all normal differentiated cells express high levels of p27, compounds interfering with p27 degradation have a high potential for tumor specificity, thus minimizing drastic adverse effects.

No data are currently available on the expression of KPC1, KPC2, and PIRH2 proteins in cancer tissues since they were discovered more recently. However, their involvement in p27 degradation coordinately with SKP2 suggests an equal potential of these enzymes as cancer drug targets. A survey of the Oncomine database (www.oncomine.org), which compiles and mines a wide assortment of published expression microarray data (>25,000 arrays) for statistically significant differences, revealed that PIRH2 mRNA is overexpressed in prostate cancer. Likewise, rises in KPC2 mRNA correlate with increased malignant grade (Gleason score) in this disease. The changes identified are similar in extent to those noted for SKP2 mRNA. Other malignancies overexpressing PIRH2 mRNA include Burkitt lymphoma, diffuse large B cell lymphoma, and cervical cancer, whereas KPC2 mRNA is overexpressed in lung carcinoids, bladder cancer, acute myeloid leukemia, several lymphomas, and multiple myeloma. Although KPC1 mRNA was not found overexpressed in prostate cancer, it is strongly increased in ovarian cancer. These data potentially implicate ectopic activation of all three pathways of p27 degradation in prostate cancer and other malignancies.

### The UPS as a Cancer Drug Target

Due to its implication in an ever-increasing number of diseases, the UPS is the subject of intense interest in the drug discovery arena (Nalepa et al., Nat. Rev. Drug Discov. 5:596-613, 2006). Efforts focus on inhibiting particular enzymes of the ubiquitylation cascade such as E2s, E3s, and deubiquitylating enzymes. The only FDA approved drug targeting the UPS is the proteasome inhibitor bortezomib (Velcade, Millenium Pharmaceuticals, Inc.). Despite its indiscriminate activity toward UPS substrates, Velcade has shown a surprising preference in killing tumor cells over normal cells, thereby advancing to a first line defense against multiple myeloma (Richardson et al., Annu. Rev. Med. 57:33-47, 2006). Toxicity can, however, be severe, and the drug has met with limited success in several solid tumors, including prostate cancer (Morris et al., J. Urol. 178:2378-2384, 2007; Greco, Clin. Genitourin. Cancer 5:278-283, 2007; Dreicer et al., Clin. Cancer Res. 13:1208-1215, 2007). Small molecules that target a more restricted set of UPS functions are therefore highly desirable.

Many drugs target a protein receptor and lead either to its inhibition or activation. If a selective binder is identified, this in no way guarantees biological activity, as the compound must meet many requirements such as solubility, absorption, excretion, and favorable tissue distribution. A considerable medicinal chemistry effort is therefore required to improve pharmacological features (Kuruvilla et al., Nature 416:653-657, 2002).

At least the problem of cell permeability is a priori addressed in a cell-based phenotypic screen. Here, compound libraries are applied directly to cells and changes are recorded that result from interference with complex pathways (Stockwell et al., Chem. Biol. 6:71-83, 1999). This differs from in vitro screening approaches for a preselected target without the immediate concern for the compound's effect within whole cells. Thus, cell-based screening can be used to affect a selected physiological endpoint without prior knowledge of the target.

The emerging complexity of the p27 degradation pathway, which involves three cooperating E3s (SCF^{SKP2}, KPC, PIRH2) and two of which are composed of multiple subunits, lends itself to the cell-based screening format, which we have chosen for our initial work. Rather than focusing on a single component of an enzymatic complex, we have asked whether cell-permeable small compounds can be identified that interfere with p27 degradation in prostate cancer cells. In other words, we were asking whether ectopic p27 degradation in cancer cells is amenable to inhibition by small molecules (“druggable”). Of the 7,368 compounds which were screened at least in duplicate, SMIP004 upregulated p27 in prostate cancer cells, inhibited p27 degradation, preferentially killed prostate cancer cells compared with normal human fibroblast, was readily soluble, had a low molecular weight, lacked obvious unspecific reactive groups, displayed multiple sites available for chemical derivatization, and was confirmed by secondary screening.

While compound SMIP004 has provided proof-of-principle that p27 depletion in cancer cells is “druggable”, a subsequent medicinal chemistry effort is required to improve potency to a level that will facilitate target identification and testing for anti-cancer activity in a rodent in vivo model for prostate cancer.

### Characterization of the LNCaP-S14 Screening Cell Line

The cell-based screen relied on the detection of p27 by immunofluorescence in a derivative of the human prostate cancer cell line LNCaP constitutively expressing Myc-tagged SKP2 at approximately one-fold excess over endogenous SKP2 (LNCaP-S14 cells). Stable expression of SKP2 led to maximum downregulation of basal p27 levels. This recapitulates the situation prevailing in the majority of primary human prostate cancers (De Marzo et al., Am. J. Pathol. 153:911-919, 1998; Cheville et al., Mod. Pathol. 11:324-328, 1998; Cote et al., J. Natl. Cancer Inst. 90:916-920, 1998; Yang et al., Clin. Cancer Res. 8:3419-3426, 2002).

Based on this finding, we developed a microtiter plate assay to screen for compounds that can reverse p27 downregulation in these cells according to the procedure proposed in the original application of this grant. To this end, we first asked whether p27 downregulation in S14 cells was also apparent by immunofluorescence staining. S14 cells showed a strong reduction of nuclear p27 staining, whereas SKP2 staining was increased. Thus, the immunofluorescence staining closely mirrored total p27 and SKP2 protein levels determined by immunoblotting in S14 cells.

We next explored the possibility to restore p27 levels in S14 cells by exposure to small molecules. Steady-state levels of p27 are maximally downregulated in S14 cells, but are restored upon the addition of proteasome inhibitors (MG132, epoxomycin). This strongly suggests that p27 downregulation in S14 cells occurs, at least in part, through SKP2-driven proteolysis. The CDK inhibitor roscovitine, which is undergoing phase II clinical trials for diverse cancers, also upregulated p27 in S14 cells. This finding is consistent with the known requirement of CDK2 activity for p27 degradation (Carrano et al., Nature Cell Biol. 1:193-199, 1999; Tsvetkov et al., Curr. Biol. 9:661-664, 1999; Sheaff et al., Genes Dev. 11: 1464-1478, 1997; Vlach et al., EMBO J. 16:5334-5344, 1997; Montagnoli et al., Genes Dev. 13:1181-1189, 1999). The inhibition of p27 degradation by proteasome and CDK inhibitors was also apparent by immunofluorescence staining (Lu et al., BMC Cell Biol. 3:22, 2002). This demonstration was important since it provided the method of detection in the cell-based screen described below.

### Optimization and Validation of the Screening Protocol

To develop a screening assay, the immunofluorescence assay was adapted to 384-well plate format. All parameters, including the number of cells to be plated, fixation and blocking conditions, antibody concentrations, and incubation times with compounds were extensively optimized using positive (proteasome inhibitors, roscovitine) and negative controls (vehicle). In parallel with the efforts described above, we established the following reliable protocol: 4000 S14 cells per well were seeded into 384-well plates in 30 μL RPMI and 10% FBS using an automated liquid handler (Wellmate). After 24 h, 40-100 nL of positive control compound was pin-transferred, followed by incubation for 16-18 h. Cells were fixed by adding 20 μl of 10% paraformaldehyde (final concentration 4%) directly to the medium and incubation for 20 minutes at RT. Cells were permeabilized by washing 3×5 min with PBS, 0.1% Triton X100 (30 μL/well), followed by blocking in 30 μL/well 5% nonfat dry milk in PBS, 0.1% Triton X100 for 1 hour at RT. Cells were incubated in 15 μL/well primary antibody (anti-p27, Transduction Laboratories K25030, 1:1000 in blocking buffer) for 1 h. Following one wash in 30 μL/well blocking buffer, 15 μL/well of secondary antibody (Alexa Fluor 594 goat anti-mouse, Invitrogen A11005, 1:200 in blocking buffer) was added for 1 h. Cells were washed 3×5 min in PBS, 0.1% Triton X100 (30 μL/well) and stained with Hoechst dye for 2 min at RT. Cells were washed twice in PBS, 5 min each. 30 μL/well of PBS were added, and plates were sealed and stored in the dark at 4 C.° until scanning.

Plates were scanned in an ImageXpress automated microscope at dual wavelength to detect Hoechst and Alexa Fluor 594. Two images per well were obtained at each wavelength and analyzed with MetaXpress software. Pixel dimensions for detection of cell nuclei were set to 7×12, a setting that performed well in the detection of nuclear staining, while successfully excluding aggregates of cells growing on top of each other. Cells were scored positive for Hoechst, if the integrated pixel intensity was 210-fold above local background, and positive for p27 when staining was 30-fold above background (signal obtained with secondary antibody). MetaXpress processing of the raw images provided quantitative measures of the total cell number and the number of p27-positive cells in a given field.

To evaluate the quality of our assay conditions, we determined the Z′-factor for the positive control reagent roscovitine. The Z′-factor measures the variation and separation bands of an assay and is defined as:

$${Z}^{′} = 1 - \frac{( 3 {SD}_{+} + 3 {SD}_{-} )}{\vert {Ave}_{+} - {Ave}_{-} \vert}$$

where “SD” refers to the standard deviation, “Ave” refers to the average staining intensity in all recorded wells, and “+” and “−” refer to positive and negative controls, respectively (Zhang et al., J. Biomol. Screen. 4:67-73, 1999). We determined a Z′-factor of 0.48 for our assay, which is appropriate for a successful screen (Zhang et al., J. Biomol. Screen. 4:67-73, 1999).
Results from Primary and Secondary Screens for SMIPs

Using the above protocol, we have screened 7,368 compounds (derived from a variety of different compound libraries) in duplicate. Compound concentrations varied from 17-80 μM, depending on molecular weight. Each screening plate also contained positive and negative controls. Instead of recording percent p27-positive cells, we determined the number of standard deviations by which a signal for a given compound differed from the mean signal of the entire plate. This number is known as the z score and facilitates the comparison of results across a series of different plates and experiments.

In the initial primary screen, we identified 249 compounds that upregulated p27 levels in S14 cells. Reassuringly, the screen re-isolated our positive control MG132. In addition, the PI3 kinase inhibitor Wortmannin scored positive. The latter finding is consistent with the known role of PI3K signaling in promoting SKP2-mediated p27 down-regulation (Zhang and Wang, Oncogene 25:2615-2627, 2006; Bashir et al., Nature 428:190-193, 2004). Upon manual examination of microscopic images, 21 duplicate compounds (occurring in more than one library), 7 false positives (DNA stains), and 45 compounds where the staining was of low quality (few cells left in well, background, indiscriminate whole cell staining) were eliminated. This resulted in a list of 176 candidate SMIPs that were grouped into the categories weak, medium, strong, based on their z scores. Strong hits were defined as having a z score of >5, medium hits a z score of 3-5, and weak compounds a score <3. At a z score of 3, a compound has a theoretical probability of 0.00135 to be a false positive hit. Further statistical analysis (cumulative z scores of wells across all plates) demonstrated that we did not incur errors caused by plate position of compounds.

One hundred and eighteen strong and medium compounds (z>3) were further examined by secondary screening in LNCaP-S14. Secondary screening was performed in duplicate and at three different concentrations. Altogether, dose-dependent SMIP activity was confirmed for 60 compounds.

### Hit Prioritization

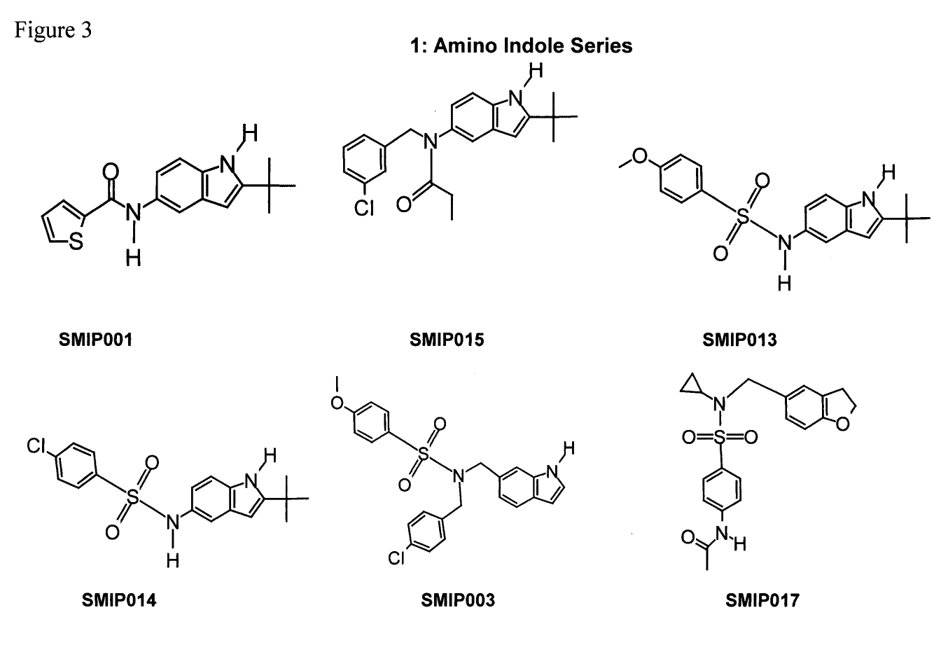

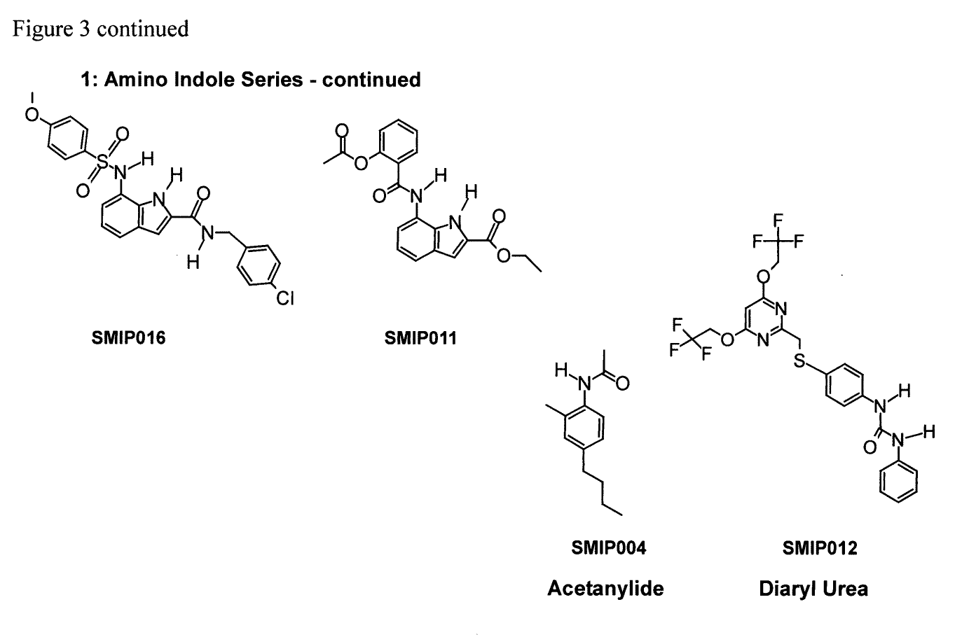

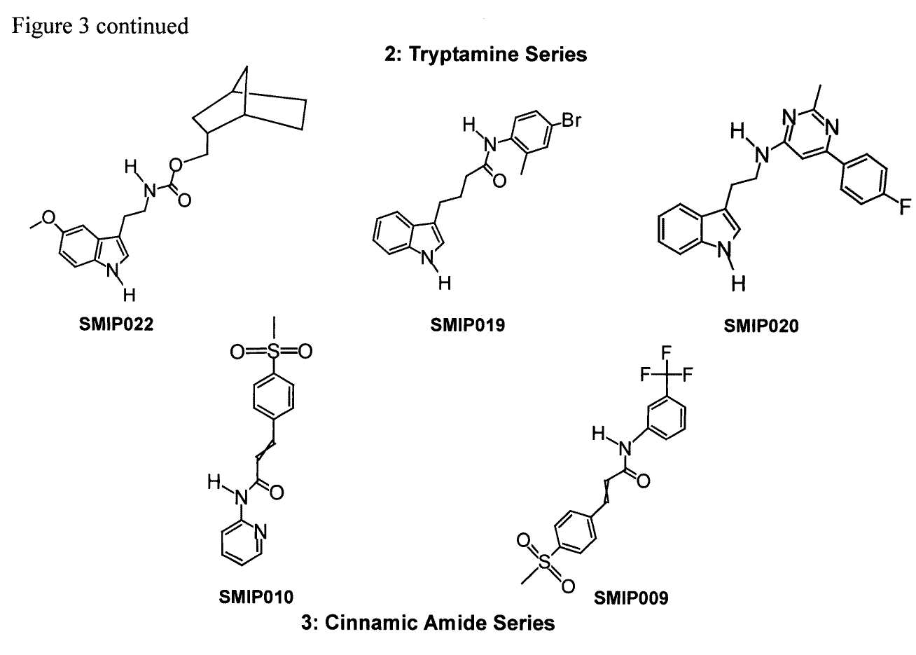

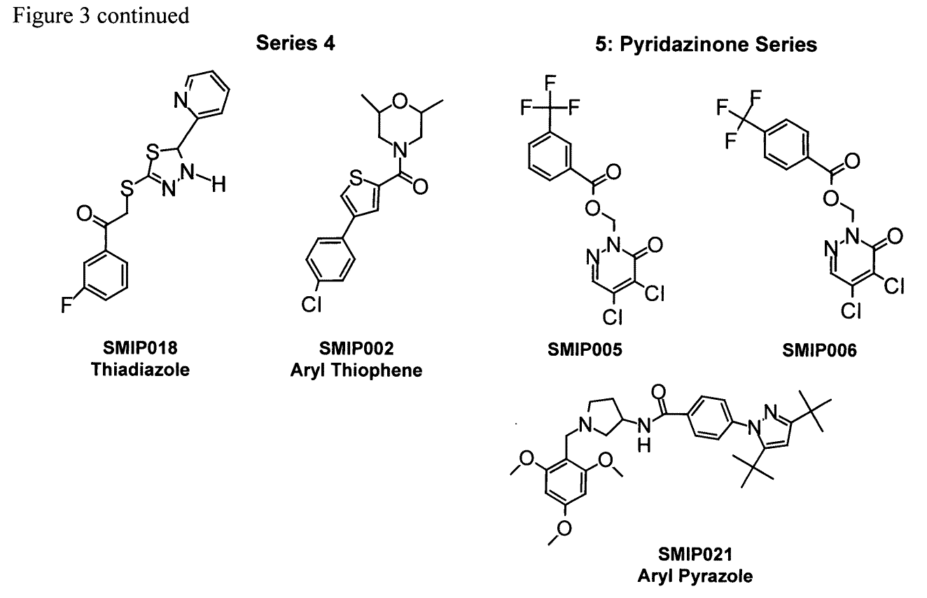

We selected 20 SMIPs for further analysis. The selection was based on chemical properties (molecular weight, number of H bond donors and acceptors, calculated solubility). These 20 SMIPs were tentatively assigned to one of eight structurally related groups (FIG. 3); 17 SMIPs were commercially available and purchased as powders (SMIP020-022 were discontinued by the manufacturer). The compounds were dissolved in DMSO, verified by HPLC-MS, and subjected to further validation experiments as described below. SMIP014 was removed from our list at this point, because it unspecifically stained DNA, thus leaving 16 compounds for further study.

### Further Characterization of SMIPs

We next performed a dose-response experiment using the immunofluorescence (IF) screening assay. Cells were treated with increasing concentrations of SMIPs for 16 hours, followed by immunostaining for p27. Table 3 shows the accumulation of p27 (fold-induction normalized to the staining obtained with DMSO) upon treatment with the SMIPs. As shown in Table 3, only 12 of the 16 purchased SMIPs induced p27>2-fold at the maximum dose. This suggests that four of the original hits from the screening library may have been contaminated with other bioactive molecules. Regardless, several SMIPs showed a robust upregulation of p27 in S14 cells.

TABLE 3
Dose response of SMIP activity in LNCaP-S14 cells.

| SMIP | 0 | 1 | 5 | 10 | 20 | 40 |
|---|---|---|---|---|---|---|
| 001 | 1.00 | 0.94 | 1.21 | 1.74 | 2.16 | 2.90 |
| 002 | 1.00 | 0.15 | 0.31 | 0.36 | −0.16 | 0.96 |
| 003 | 1.00 | 0.38 | 0.54 | 0.99 | 1.83 | 3.18 |
| 004 | 1.00 | 0.49 | 0.51 | 1.45 | 3.99 | 5.16 |
| 005 | 1.00 | 0.02 | 1.76 | 3.23 | 3.93 | 7.26 |
| 006 | 1.00 | 0.64 | 0.86 | 0.88 | 2.30 | 7.54 |
| 009 | 1.00 | 0.04 | 0.07 | −0.02 | 0.39 | 0.98 |
| 010 | 1.00 | 0.71 | 0.47 | 0.35 | 0.60 | 4.28 |
| 011 | 1.00 | 0.14 | 2.28 | 3.80 | 5.65 | 3.31 |
| 012 | 1.00 | 0.42 | 2.17 | 1.95 | 3.30 | 2.43 |
| 013 | 1.00 | −0.33 | −0.10 | 0.39 | 2.74 | 3.72 |
| 015 | 1.00 | 0.08 | 0.35 | 0.43 | 4.75 | 4.25 |
| 016 | 1.00 | 0.94 | 1.81 | 1.73 | 2.62 | 2.44 |
| 017 | 1.00 | 0.58 | 0.40 | 0.54 | 0.50 | 1.31 |
| 018 | 1.00 | 0.26 | 0.71 | 0.67 | 0.92 | 3.07 |
| 019 | 1.00 | 0.93 | 0.86 | 1.08 | 1.16 | 1.20 |

The 16 SMIPs were also tested in another prostate cancer cell line, DU145, with similar results (not shown). Importantly, none of the active SMIPs led to accumulation of NKX3.1, another unstable protein that is degraded by the UPS. This suggested that active SMIPs are not general inhibitors of protein degradation.

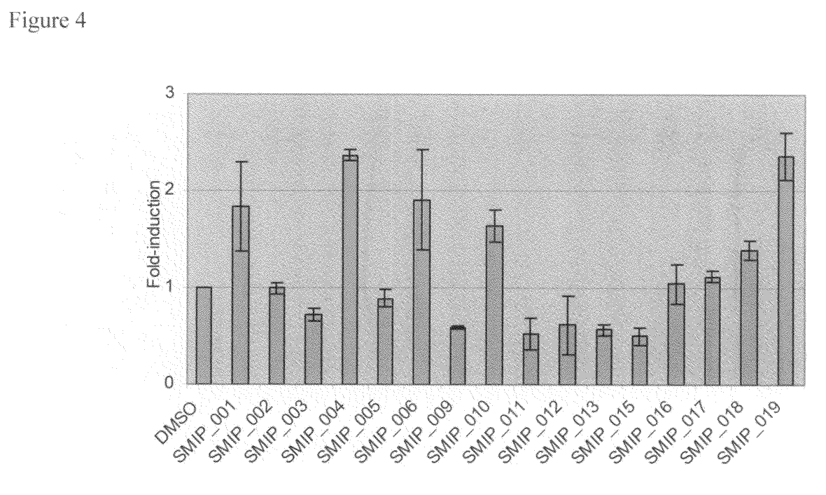

Next, we assessed the effect of SMIPs on p27 by immunoblotting (IB). S14 cells were incubated with a single dose of SMIPs (40 μM) and p27 levels were determined relative to the DMSO vehicle control. Tubulin reactivity was used as a reference. Most, but not all SMIPs active in the IF assay were also active in the IB assay (FIG. 4). However, the effects were generally weaker, an observation that we had made previously during assay development (with positive control compounds roscovitine, MG132). This is not surprising, since the two assays are based on different metrics: IF measures percent cells positive for p27 above a certain threshold, IB measures the total amount of p27 that can be extracted from a cell population relative to a reference. In any case, SMIPs 001, 004, 006, 010, and 019 caused approximately two-fold p27 upregulation in duplicate experiments. While seemingly small, such an increase is biologically significant because p27 levels do not vary more than 2 to 3-fold during a normal cell cycle (Zhang and Kaelin, Meth. Enzymol. 399:530-549, 2005; and our own observation). Likewise, a three-fold increase in p27 leads to efficient growth arrest of LNCaP cells (Lu et al., BMC Cell Biol. 3:22, 2002).

### Effect of SMIPs on Cell Viability and Cell Cycle Progression

We next explored the effects of SMIPs on cell viability. S14 and IMR90 cells were exposed to increasing concentrations of SMIPs for 72 h and scored for viability using the MTT assay. IC₅₀ values (concentration of 50% growth inhibition) were calculated (Table 4). Based on the comparison to IC₅₀ data obtained with IMR90 normal human fibroblasts (Table 4), we selected SMIPs 001, 004, and 010 for further analysis, because they displayed the highest specificity for killing S14 prostate cancer cells. Although SMIP005 also showed good specificity, it was excluded at this point due to structural features that suggested potential unspecific reactivity with cellular macromolecules.

TABLE 4
SMIP cytotoxicity

| | LNCaP-S14 | IMR90 |
|---|---|---|
| MG132 | 0.379 | 0.445 |
| Thapsigargin | 0.0078 | 0.0311 |
| SMP001 | 4.63 | >20 |
| SMP002 | 11.09 | >40 |
| SMP003 | 15.92 | 65.36 |
| SMP004 | 1.091 | >20 |
| SMP005 | 1.709 | 17.38 |
| SMP006 | 6.63 | NT |
| SMP009 | >40 | >40 |
| SMP010 | 3.56 | >40 |
| SMP011 | >40 | ~40 |
| SMP012 | 4.59 | ~10 |
| SMP013 | 18.15 | ~20 |
| SMP015 | 13.02 | ~20 |
| SMP016 | 14.63 | 4.41 |
| SMP017 | 23.73 | >40 |
| SMP018 | >40 | >40 |
| SMP019 | NT | ~40 |

We next compared IC₅₀ values for cytotoxicity of SMIP004 in S14 compared with parental LNCaP cells. IC₅₀s were generally lower in S14 cells, suggesting that high SKP2 levels may sensitize cells to SMIP004 (Table 5). We are testing whether SMIP-induced cytotoxicity is due to apoptosis.

TABLE 5
IC50 values for SMIP004 in S14, LNCaP, and IMR90 cells

| Cell line | 72 h | 96 h |
|---|---|---|
| LNCaP-WT | 40 μM | 2.4 μM |
| LNCaP-S14 | 0.85 μM | 0.97 μM |
| IMR90 | NE | NE |

NE = no effect

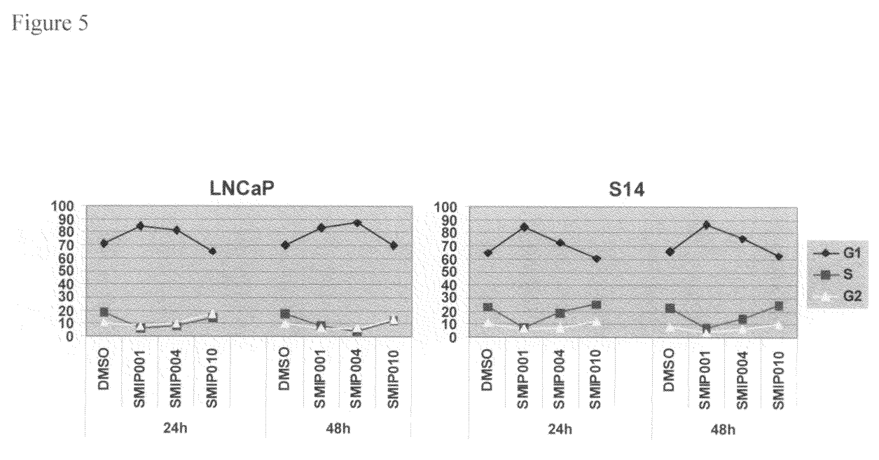

Finally, we determined the effect of SMIPs on cell cycle distribution. S14 and LNCaP cells were treated with 40 μM compound and harvested for flow cytometric measurement of DNA content after 24 h and 48 h. At both time points and in both cell lines, SMIPs 001 and 004 induced a cell cycle arrest in G1 at the expense of the S and G2 phases (FIG. 5). In contrast, SMIP010 did not alter cell cycle distribution at the doses and times applied in this experiment (FIG. 5).

### Effect of SMIPs 001, 004, and 010 on p27 Levels and Stability

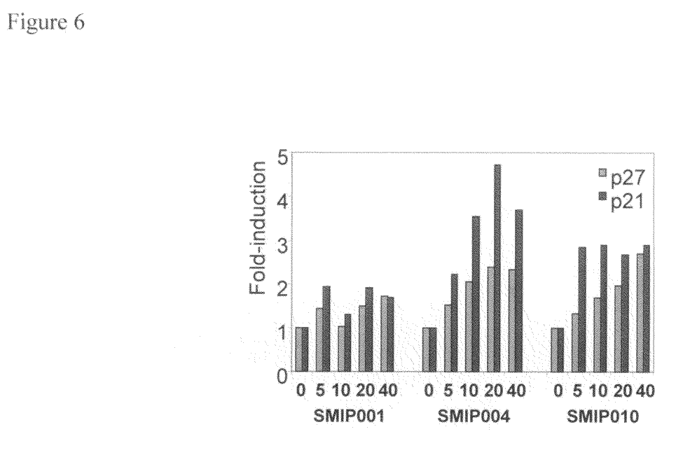

In subsequent IB experiments performed in duplicate, we assessed the dose responses for the 3 selected SMIPs. S14 cells were treated for 16 h with increasing concentrations of the respective SMIPs, followed by immunoblotting for p27 and p21. Effective concentrations (EC₅₀) ranged between 5 and 20 μM (FIG. 6). Significantly, p21, another CDK inhibitor targeted for degradation by SKP2 (Bornstein et al., J. Biol. Chem. 278:25752-25757, 2003), was also upregulated (FIG. 6). None of the compounds upregulated the levels of NKX3.1, another unstable protein that is degraded through the UPS (Guan et al., J. Biol. Chem. 283:4834-4840, 2008; Li et al., Mol. Cell. Biol. 26:3008-3017, 2006; and our observations).

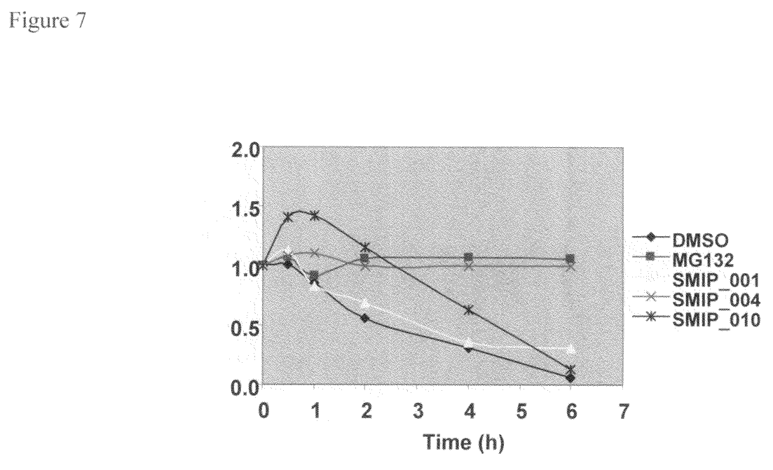

Next, we sought to test whether active SMIPs cause p27 upregulation by inhibiting its degradation. Although our cell-based phenotypic screen was not specifically designed to enrich for this property, compounds with such activity would be of very high interest for identifying small molecules that interfere with ectopic p27 proteolysis in cancer cells. To measure p27 stability, SMIP-treated LNCaP-S14 cells were incubated with the protein synthesis inhibitor cycloheximide (CHX). Cells were harvested after various periods, and the decay of p27 was determined by immunoblotting. The half-life of p27 was 2.36 h in vehicle-treated cells, which is in agreement with published reports (e.g., Montagnoli et al., Genes Dev. 13:1181-1189, 1999; Wolf et al., Mol. Endocrinol. 6:753-762, 1992). Significantly, among the three preselected SMIPs, only SMIP004 led to stabilization of p27 similar in extent to that induced by MG132 (half-lives greater than 6 h, FIG. 7).

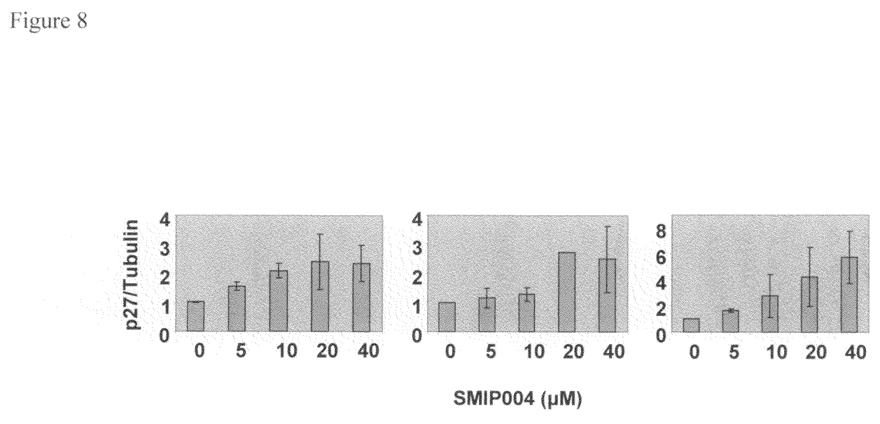

Finally, we asked whether SMIP004 could upregulate p27 in cells other than S14. S14 and parental LNCaP cells as well as cells of the prostate cancer cell line DU145 were treated with increasing concentrations of SMIP004 for 18 h. p27 levels were determined by immunoblotting and normalized to the tubulin control. Replicate dose response experiments showed that SMIP004 led to p27 accumulation in parental LNCaP cells (FIG. 8). Likewise, the drug was active in DU145 cells (FIG. 8).

Based on all its favorable properties described above, SMIP004 was chosen for further studies. SMIP004 causes robust p27 accumulation in S14 cells, which is apparently due to p27 stabilization. In addition, SMIP004 is soluble in aqueous solution, is able to penetrate cells, and exhibits specificity in killing prostate cancer cells relative to normal human fibroblasts.

### SMIP004 Downregulates the SKP2 Pathway of p27 Degradation

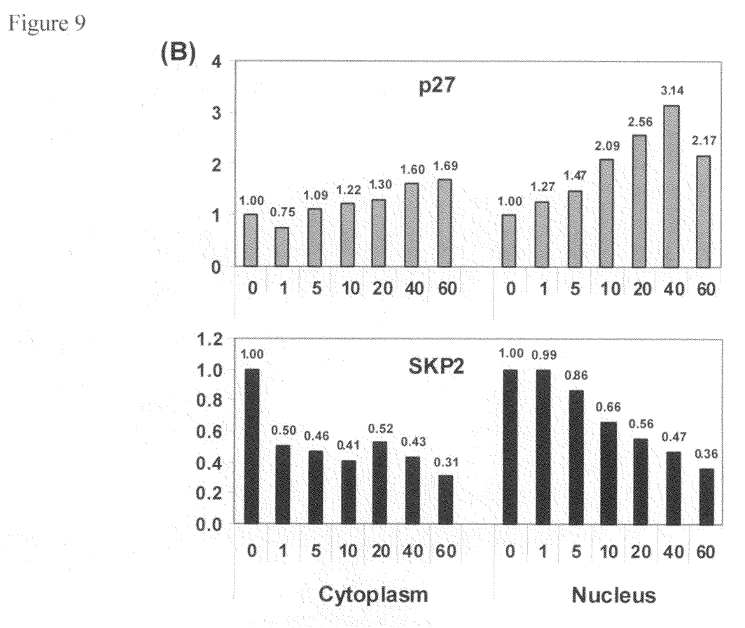

We have previously shown that androgen-mediated upregulation of p27 in LNCaP cells is due to p27 stabilization caused by downregulation of SKP2 protein and mRNA (Lu et al., BMC Cell Biol. 3:22, 2002). We therefore wondered whether SMIP004 also affects SKP2 levels. To assess this, S14 cells were treated with increasing concentrations of SMIP004 for 18 h. Cytoplasmic and nuclear protein fractions were prepared and p27 and SKP2 levels were measured by immunoblotting. For increased subcellular resolution, we also performed biochemical separation into nuclear and cytoplasmic fractions. Surprisingly, p27 was more abundant in the cytoplasmic than in the nuclear fraction as found in duplicate experiments (FIG. 9). This finding is only partly explained by the contamination of the cytoplasmic fraction with nuclear proteins as revealed by the partitioning of the nuclear marker Nucleolin (FIG. 9). Since our IF-based studies have detected p27 only in S14 cell nuclei, p27 appears to show substantial leakage into the cytoplasm during cell fractionation. Alternatively, the IF assay may not accurately represent cytoplasmic p27 levels due to the dilution effect caused by the higher volume of the cytoplasm relative to the nucleus. Cytoplasmic localization of p27 is now well-established (Dreicer et al., Clin. Cancer Res. 13:1208-1215, 2007; Stockwell et al., Chem. Biol. 6:71-83, 1999; Yang et al., Clin. Cancer Res. 8:3419-3426, 2002; Montagnoli et al., Genes Dev. 13:1181-1189, 1999). Regardless, robust, dose-dependent induction of p27 by SMIP004 was observed in both the cytoplasmic and the nuclear fractions (FIG. 9).

Like p27, SKP2 exhibited substantial partitioning into the cytoplasmic fraction, but showed a cytoplasmic and nuclear expression pattern opposite to that of p27. Both, endogenous SKP2 as well as exogenous Myc-SKP2 expressed from the stably integrated plasmid were downregulated by SMIP004 in a dose-dependent manner (FIG. 9). Quantification of the immunoblots revealed a close correlation between SKP2 and p27 levels (FIG. 9). This finding suggests that SMIP004-induced p27 stabilization is caused by the downregulation of SKP2 thus identifying the SKP2-dependent proteolysis as a candidate cellular target pathway of SMIP004. Interestingly, upregulation of p27 by the proteasome inhibitor bortezomib was very recently shown to also coincide with SKP2 downregulation (Wolf et al., Nucl. Acids Res. 23:3373-3379, 1995), although the mechanism remains unclear.

Although the p53 gene is wildtype in LNCaP cells, nuclear p53 protein levels were very low in S14 cells and not induced by SMIP004 (FIG. 9). This suggests that the compound neither activates the DNA damage checkpoint nor indiscriminately interferes with proteasome activity. Blotting with an antibody against Foxo1 (also reactive with Foxo4) gave a complex pattern of bands that could not be definitively assigned. If the band detected in the nuclear fraction is indeed a Foxo1 species, SMIP004 would appear to upregulate this SKP2 target (Huang et al., Proc. Natl. Acad. Sci. USA 102:1649-1654, 2005). Additional specificity controls are required, however, to validate this finding.

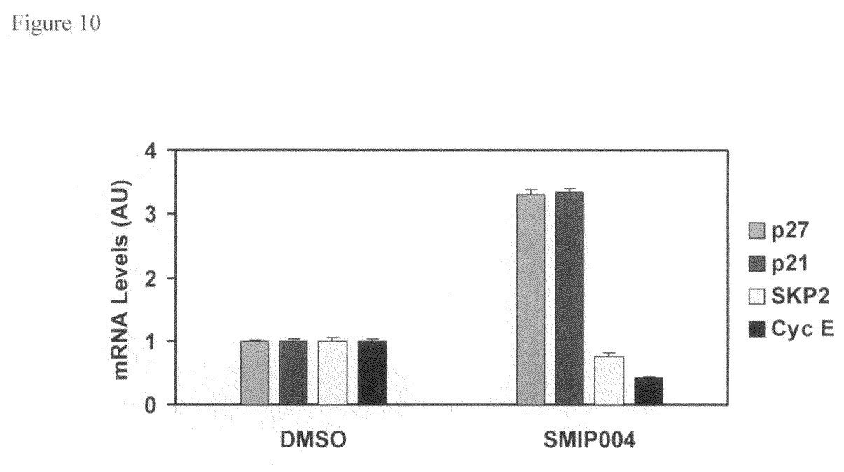

The finding that exogenous Myc-SKP2 driven by the CMV promoter was downregulated by SMIP004 suggested a posttranscriptional mechanism of SKP2 suppression. However, mRNA analysis by quantitative real-time PCR showed that SMIP004 down-regulated SKP2 mRNA (FIG. 10). This suppression appears to affect both the endogenous and the exogenous SKP2 versions, because the proteins derived from both alleles were down-regulated by SMIP004 (FIG. 9). While unexpected, the effect on exogenous SKP2 is probably due to the well-established but often underappreciated cell cycle dependence of the “constitutive” CMV promoter (Brightwell et al., Gene 194:115-123, 1997; de Boer et al., Biotechnol. Lett. 26:61-65, 2004). This promoter is much more active in the S phase than in other cell cycle phases, suggesting that it is subject to some of the same transcription control mechanisms that drive S phase genes such as SKP2. Significantly, both the SKP2 and CMV promoter contain E2F binding sites, which, in the case of SKP2, are known to be functionally significant (Terstappen et al., Nat. Rev. Drug Discov. 6:891-903, 2007; Bach et al., J. Biol. Chem. 280:31208-31219, 2005).

Contrary to SKP2 mRNA, p27 mRNA was upregulated by SMIP004, a finding that would be consistent with stabilization of Foxo1, which is an activator of p27 transcription (Medema et al., nature 404:782-787, 2000). Through its suppression of SKP2 expression, SMIP004 therefore appears to increase both p27 transcription and protein stability (FIG. 7). p21 mRNA was also upregulated by SMIP004, whereas cyclin E was suppressed (FIG. 10).

SKP2 mRNA is overexpressed in many cancers, including prostate cancer, although the exact mechanism is unknown in most cases. Gene amplification was observed in glioblastomas and lung cancer (Saigusa et al., Cancer Sci. 96:676-683, 2005; Yokoi et al., Am. J. Pathol. 165:175-180, 2004). A compound like SMIP004, which can suppress SKP2 mRNA expression, might have beneficial effects in these cancers.

### Preliminary Structure-Activity Studies for SMIP004

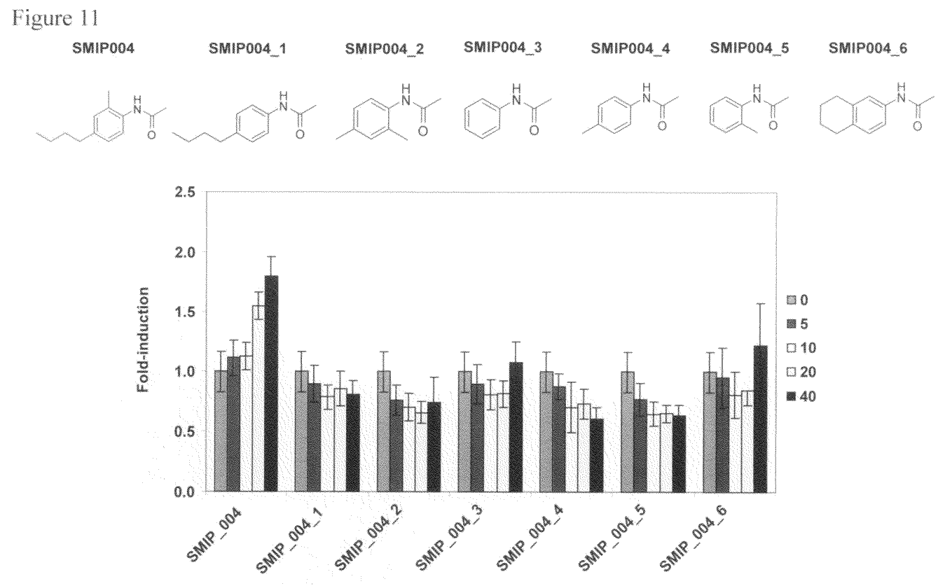

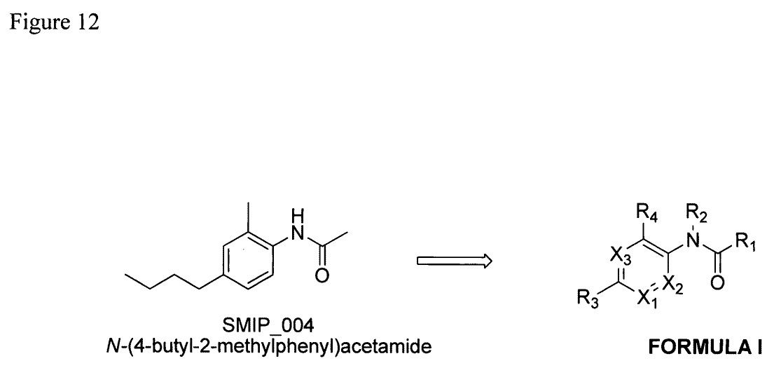

Six related SMIP004 analogs addressing various substitution points were purchased from commercial vendors. Although the selection was limited, initial structure-activity relationships (SAR) were identified. The data indicates that the alkyl chain emanating from the ring (R3 in FIG. 12) is essential for activity (FIG. 11). Likewise, the methyl group on the ring is required (FIG. 11, SMIP004_{—}1).

### EXAMPLE 3

SMIP004 was effective in the primary screen and the follow-up assays described in Example 1 at concentrations between 5-40 μM. While this range is typical for screening hits, we desired to improve the potency of SMIP004 for in vivo efficacy studies using mouse xenograft models. Likewise, we wanted to obtain a basic understanding of the bioavailability and tolerability of SMIP004 for these studies. Therefore, we are (1) optimizing the potency of SMIP004 by systematic chemical modification, (2) designing specific analogs for use as chemical probes in protein target identification studies, and (3) obtaining a basic ADME/T pharmacological profile, including in vitro and in vivo pharmacokinetic as well as toxicological information of select SMIP004 analogs.

The first goal is pursued through a structure activity relationships (SAR) analysis with the purpose of understanding which features of compound structure drive activity. The primary assay to assess improvements in EC₅₀ is the immunofluorescence assay in a 384-well format. Once the molecular pathways targeted by SMIP004 have been defined more precisely, assays with increased target specificity are employed.

### Overview of the Medicinal Chemistry Approach

The small molecule denoted as SMIP004 was selected from a series of molecular classes derived from a systematic evaluation and triage of the initial high throughput screening data set. SMIP004 demonstrated approximately a five-fold upregulation of p27 in S14 cells at a 40 μM assay concentration. Although dose-responsive, the initial hit molecule requires a boost in potency as measured by the cellular p27 accumulation assay. We are modifying the current hit to provide improved lead molecules that demonstrate at least 10-fold improved potency (3-5-fold upregulation of p27 at ≦4 μM assay concentration). We are then targeting analog synthesis towards increasing potency by another order of magnitude. Our experience indicates that single digit μM target affinity can often be sufficient for cell-based target identification efforts. SMIP004 is an ideal starting point for compound optimization, since it has a low molecular weight (205 Daltons), good physicochemical properties, and multiple points for chemical modification to improve potency. An overview of our optimization strategy is illustrated in FIG. 12. Single-point analogs are prepared that are designed to provide both specific SAR information and to define functional groups that allow for covalent trapping of the cellular protein that SMIP004 is targeting. Multi-point substitution analog arrays and libraries are also prepared in order to determine which regions of the molecular scaffold are available to provide improved potency. In the absence of a protein structure-small molecule binding hypothesis we must derive the initial SAR in a de novo manner. As areas of substitution that modulate potency are identified, physicochemical (solubility, logP, etc.) and in vitro pharmacological profiles (absorption, distribution, metabolism, excretion and toxicity [ADME/T]) are measured on selected analogs. This data assists the chemistry effort in either improving or maintaining desirable properties in parallel with the synthetic design to further improve potency. The goal is to modify SMIP004 to generate one or more bioavailable in vivo candidate analogs with superior cellular potency.

All compounds are synthesized using streamlined techniques (resin capture, SPE, polymer supported reagents, microwave acceleration, microfluidics) in vial or 48-well Bohdan MiniBlock™ synthesizers (Mettler Toledo, Columbus, Ohio). Starting materials are commercially available or are readily derived. Purification is accomplished using an automated UV-guided, mass-triggered preparative HPLC workstation (Waters Corporation, Milford, Mass.). Each analog is prepared in 5-10 mg quantity at a greater than 95% purity level. Selected analogs are pharmacologicalally evaluated as potency increases and major scaffold changes are made.

### Single-Point Analogs.

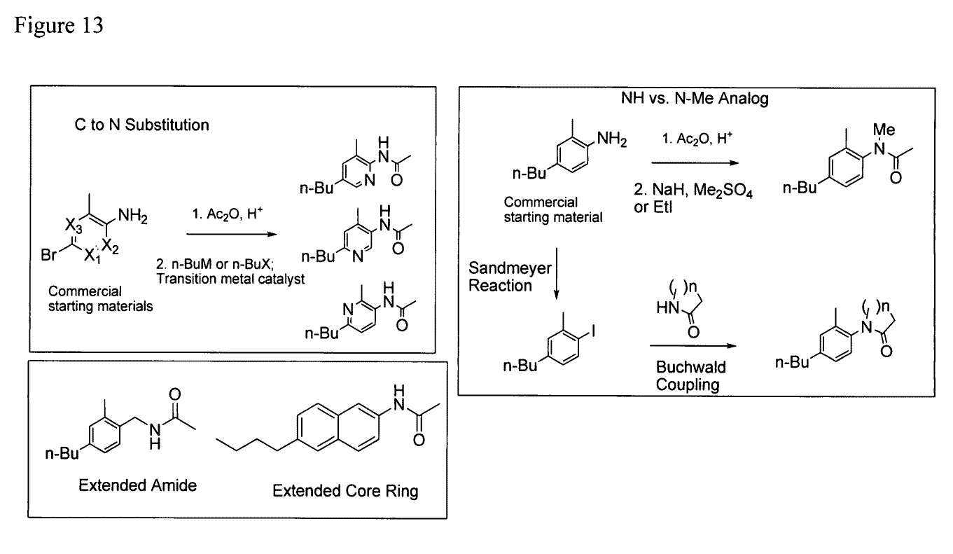

Six related SMIP004 analogs addressing R3 and R4 substitution points were purchased from commercial vendors. Although the selection was limited, initial SARs were identified. The data indicates that R3 requires an alkyl chain over methyl or hydrogen groups and R4 is inactive as hydrogen and substitution is required (FIG. 11). The synthesis of key single-point derivatives provides a wealth of SAR information and assists in the design of subsequent arrays and/or libraries. The analogs are designed to answer a specific structural question. FIG. 13 illustrates this approach. Key single-point analogs include those that determine whether substitution of the amide NH is tolerated. The corresponding N-Me, N-Et and cyclic derivatives (lactams) are prepared. Also shown is an example of heteroatom substitution. The use of a C to N exchange is often used to modulate lipophilicity and polar surface area, and provides a hydrogen-bond acceptor or donor moiety to increase interaction with protein residues (R3=n-Bu and X═N) and to modulate solubility. All three regioisomers are being explored. Data for these and other analogs are utilized in the subsequent design of scanning libraries. An example of additional scaffold structure questions is the effect of extending the amide N further from the phenyl core as well as an extension of the phenyl to a naphthyl moiety.

### R1/R2 Amide Scan Array

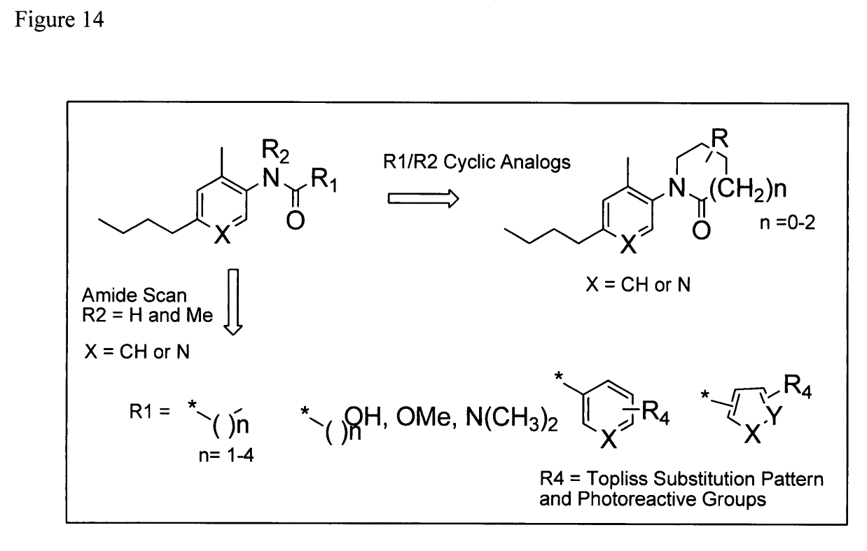

A schematic for an R1/R2 scanning array is illustrated in FIG. 14. A diverse array of R1 amides is prepared (R2=H) and the N-Me scaffold included as a second dimension if single-point data is positive. The initial target array consists of 96 analogs. The design is based on a diversity-oriented selection of substituents for R1 including aryl, heteroaryl, substituted aryl and heteroaryl rings, alkyl chain, branched, and rings, and functional group substituted alkyl moieties. Selection parameters include polar surface area, cLogP, calculated solubility, and general Lipinski parameters (Rule of Five). A representative subset of an initial planned 96 analog array is illustrated. The objective is to probe size, space, and charge at this region of the molecule. Ideally, substitutions are found that increase potency due to increased interaction with the target protein. The same principle holds true for the R3 scan library (below). The resulting SAR data drives the selection and number of the next round of analogs that are made. Typically, follow-up arrays of 12-48 additional analogs, focused on a specific SAR observation, are targeted. A second round library based on active R1/R2 cyclic lactams is prepared, again, based on initial single-point data and the effect of functional group substitution of the lactam ring is explored (12-48 analogs). As potency is increased, in vitro pharmacology assays provide data on selected analogs so properties can be monitored and corrected as required.

### R3 Lipophilic and Functional Group Scan Array

Information obtained from screening related commercial analogs indicated that an alkyl group at R3 maintained potency (FIG. 11). This suggests that lipophilic character and cellular efficacy may be related. In general, lipophilicity and polar surface area (PSA) have been demonstrated to modulate cell permeation of small molecules (Papageorgiou et al., Bioorg. Med. Chem. Lett. 11:1549-1552, 2001). Using this hypothesis, the initial 1-dimensional array is prepared primarily using cross-coupling chemistry. This assay is computationally designed to span several units of calculated logP values (SimulationsPlus ADMET Predictor v2.4.1, Simulations Plus, Inc., Lancaster, Calif.) and to provide a change in the hydrocarbon chain geometry and topology as outlined for selected examples (FIG. 15). The overall library is to consist of at least 24 lipophilic R3 substituents along with 24 additional heteroatom containing functionalized butyl or related chains/rings thus adding a dimension of polarity. As illustrated in the scheme, the X═CH to N substitution alters the lipophilic profile by a log unit.

### Combinatorial SAR Libraries

After the initial R3 and R1/R2 arrays are evaluated, a three-dimensional library is prepared incorporating the most favored X, R1, R2, and R3 functional groups in a combinatorial manner (X1-3×R1/R2×R3). The objective of generating and testing a library is to capitalize on each contributing SAR vector, functionality and improved protein interactions and test whether, in combination, the effect is additive thus providing an analog with superior potency.

Chemical Pathways and Conceptual Approach to Target Identification

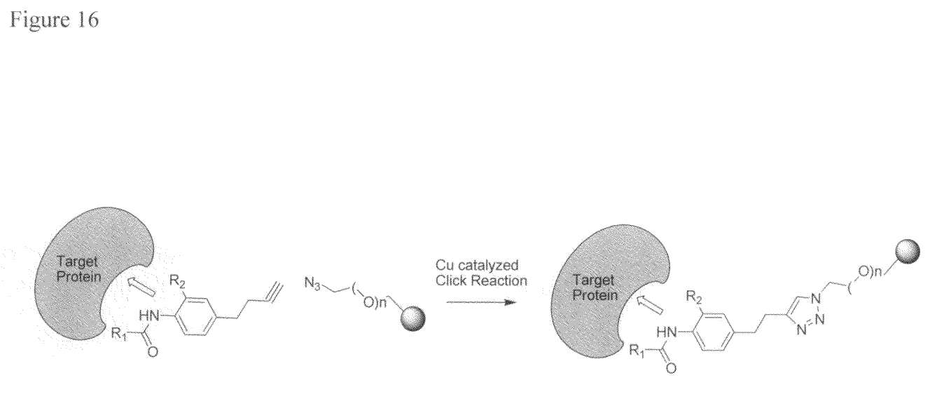

Through empirical observation, it is felt that a suitable affinity probe should be within 50-100 fold of the activity of a compound that has best efficacy in the cellular assay. This is only one of several factors since the concentration of the putative target protein also is important. The design of orthogonally reactive probe molecules is driven by the SAR studies (Peddibhotla et al., J. Am. Chem. Soc. 129:12222-12231, 2007). An example and conceptual design of a probe is shown in FIG. 16. A two-step process for derivatization was selected. This maximizes the probability that biological activity is maintained over simply tethering the molecule to a solid support prior to a protein cross-linking reaction. The incorporation of an alkyne moiety allows for exploitation of the ‘click’ triazine reaction. With this, the small molecule-protein complex can either be pulled-down via an azide cycloaddition to a solid support (shown in FIG. 16) or a tethered fluorophore can be attached for subsequent cell imaging. The inclusion of photochemical or transition metal catalyzed protein reactive/affinity immobilization functionalities during the SAR process allows for the identification of the proposed affinity probes. Protein reactive functional groups (e.g. azide, chloromethlyketone, trifluoromethyldiazarine, or benzophenone at R1 or R2) and affinity linker groups (alkyne) are included in single-point analogs as the emerging R1/R2 and R3 SAR dictates.

All publications, patents and patent applications are incorporated herein by reference. While in the foregoing specification this invention has been described in relation to certain preferred embodiments thereof, and many details have been set forth for purposes of illustration, it will be apparent to those skilled in the art that the invention is susceptible to additional embodiments and that certain of the details described herein may be varied considerably without departing from the basic principles of the invention.

What is claimed is:

1. A method to identify a small molecule inhibitor of p27 depletion comprising: (a) contacting a mammalian cell with a test agent; and (b) determining whether the test agent inhibits ubiquitin-dependent proteolysis of p27 in the mammalian cell.

2. The method of claim 1 wherein the mammalian cell has substantially lower than normal levels of p27.

3. The method of claim 2 wherein the mammalian cell overexpresses myc-SKP2.

4. The method of claim 2 wherein the mammalian cell is an LNCaP-S14 cell.

5. The method of claim 1 comprising determining whether the test agent inhibits ubiquitin-dependent proteolysis of p27 in the mammalian cell by determining levels of p27 in the cell.

6. The method of claim 5 comprising contacting the mammalian cell with an antibody that is specific for p27 and measuring binding of the antibody to p27 in the cell.

7. The method of claim 6 wherein the antibody comprising a fluorophore and binding of the antibody to p27 in the cell is measured by measuring immunofluorescence of the mammalian cell.

8. The method of claim 1 further comprising contacting a cancer cell with the test agent and determining whether the test agent inhibits ubiquitin-dependent proteolysis of p27 in the cancer cell, wherein the cancer cell is different than the mammalian cell.

9. The method of claim 8 wherein the cancer cell is selected from the group consisting of a prostate cancer cell, a cervical cancer cell, and a breast cancer cell.

10. The method of claim 8 wherein the cancer cell is selected from the group consisting of a HeLa cell and an MCF7 cell.

11. The method of claim 8 comprising determining whether the test agent inhibits ubiquitin-dependent proteolysis of p27 in the cancer cell by determining levels of p27 in the cancer cell.

12. The method of claim 8 comprising determining whether the test agent inhibits ubiquitin-dependent proteolysis of p27 in the cancer cell by inhibiting growth of the cancer cell or causing the death of the cancer cell.

13. The method of claim 8 wherein the cancer cell is a human cancer cell.

14. The method of claim 1 wherein the mammalian cell is a human cell.

15. The method of claim 1 comprising providing the mammalian cell in a well of a multi-well plate.

16. An automated method of claim 1.

17. A compound that inhibits ubiquitin-dependent proteolysis of p27 in a mammalian cell.

18. The compound of claim 17 that is a compound of Formula I: 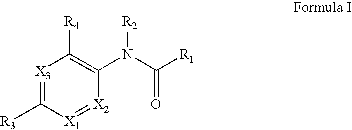
wherein:
R₁ is H, optionally substituted C1-C7 alkyl optionally comprising one or more heteroatoms, wherein such heteroatoms are each O, S, or NRₓ, where R_{x is} C1-C7 alkyl, and also optionally comprising one or more alkenyl or alkynyl bonds in the chain or ring;
R₂ is H, C1-C7 alkyl optionally comprising one or more heteroatoms, wherein such heteroatoms are each O, S, or NRₓ, where R_{x is} C1-C7 alkyl, and also optionally comprising one or more alkenyl or alkynyl bonds; and wherein R₁ and R₂ optionally form a C4-C6 ring;
R₃ is C1-C8 alkyl optionally comprising one or more alkenyl or alkynyl bonds in the chain or ring; C2-C8 aryl; or C2-C8 heteroaryl;
R4 is H, C1-C6 alkyl, or halo; and
X₁, X₂ and X₃ are each CH or N.

19. The compound of claim 18 wherein, if one of R₁ or R₂ is H, then the other of R₁ or R₂ is optionally substituted alkyl optionally comprising one or more heteroatoms, wherein such heteroatoms are each O, S, or NRₓ, where R_{x is} C1-C6 alkyl or C3-C7 cycloalkyl; and also optionally comprising one or more alkenyl or alkynyl bonds.

20. The compound of claim 17 that is a compound of Formula II: 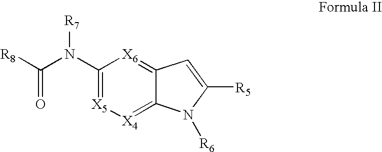
wherein:
R₅ is optionally substituted alkyl;
R₆ and R₇ are each H or Me;
R₈ is a five- or six-member optionally substituted aryl or optionally substituted heteroaryl; and
X₄, X₅ and X₆ are each CH or N.

21. The compound of claim 17 that is a compound of Formula III: 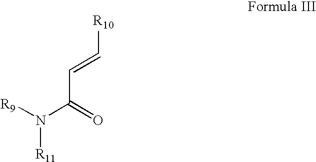
wherein:
R₉ is H or Me; and
R₁₀ and R₁₁ are each optionally substituted aryl or optionally substituted heteroaryl.

22. The compound of claim 21 wherein R₁₀ is optionally substituted phenyl.

23. The compound of claim 21 wherein R₁₁ is selected from the group consisting of optionally substituted phenyl and optionally substituted six-member monocyclic heteroaryl.

24. The compound of claim 17 that is a compound of Table 1.

25. A composition comprising an effective amount of a compound of claim 17 and a pharmaceutically acceptable carrier.

26. A method of inhibiting p27 depletion in a mammalian cell comprising contacting the mammalian cell with an effective amount of a composition comprising a compound of claim 17.

27. A method of treating a cancer in a patient in need of such treatment comprising administering to the patient an effective amount of a composition comprising a compound of claim 17.
